(19) United States
(12) Patent Application Publication
(10) Pub. No.: US 2002/0187514 A1
(43) Pub. Date: Dec. 12, 2002

(54) Identification of molecular targets useful in treating substance abuse and addiction

(76) Inventors: Hao Chen, Columbia, MD (US); David M. Manyak, Ellicott City, MD (US)

Correspondence Address: Finnegan, Henderson, farabow,, Garrett & Dunner, L.L.P., 1300 I Street, N.W., Washington, DC, 20005-3315, US

(21) Appl. No.: 10/105,407

(22) Filed: Mar. 26, 2002

Related U.S. Application Data
(63) Continuation-in-part of application No. 09/558,232, filed on Apr. 26, 2000.
(60) Provisional application No. 60/130,992, filed on Apr. 26, 1999.

(51) Int. Cl. G01N 33/53; G01N 33/542; G06F 19/00; G01N 33/48; G01N 33/50

(52) U.S. Cl. 435/7.1; 435/7.9; 702/19

(57) ABSTRACT

The invention provides methods determining a set of one or more molecular targets for developing a treatment for abuse of, or addiction to, a substance. The methods involve determining a biological activity profile by determining a set of molecular targets whose activity is effected by the abused or addictive substance. The biological activity profile may then be used in other methods of the invention to identify at least one chemical compound to treat abuse or addiction. The chemical compounds interact with the molecular targets in a manner substantially the same as the abused or addictive substance. The invention also provides methods for treating substance abuse wherein chemical compounds identified by the methods of the invention are administered in effective amounts to patients in need thereof. A computer system for implementing the methods of the invention is also provided.

This application is a continuation-in-part of U.S. patent application Ser. No. 09/558,232, filed Apr. 26, 2000, which claims the benefit of U.S. provisional application No. 60/130,992, filed Apr. 26, 1999, and a continuation-in-part of U.S. provisional application No. ______, for Drug Discovery Method and Apparatus, filed Mar. 25, 2002, which are incorporated by reference herein.

The U.S. Government has a paid-up license in this invention and the right in limited circumstances to require the patent owner to license others on reasonable terms as provided for by the terms of Grant No. 1R43DA13353-01 awarded by the Department of Health and Human Services.

### BACKGROUND OF THE INVENTION

A. Field of the Invention

The present invention relates generally to a combination of chemoinformatics and bioinformatics and data on chemical-molecular target interactions to create multi-dimensional databases. More particularly, this invention relates to databases comprising chemical compound, molecular target, and biological or clinical information in which patterns or relationships of interactions between chemical compounds and molecular targets are determined and compared with other information in the database in order to draw conclusions that are useful for drug discovery and development and for related areas.

The present invention also relates to methods for determining a biological activity profile for an abused or addictive substance. A biological activity profile is a subset of molecular targets whose activity is affected by the abused or addictive substance, as determined by testing for the interaction of the substance with each of a broader set of molecular targets. The biological activity profile is useful in methods for identifying a set of molecular targets that serve as a guide for the design of therapeutic regimens and for the development of new treatments and therapeutics for treating substance abuse and addiction. For example, the biological activity profile for cocaine includes as molecular targets the dopamine transporter (“DAT”), serotonin transporter (“SERT”), and norepinephrine (also known as noradrenaline) transporter (“NET”).

The invention also makes use of systematic physiological information pertaining to chemical compounds. In the context of cocaine addiction, for example, partial inhibition of the noradrenaline transporter by cocaine is correlated with, and contributes to, dangerous cardiovascular side effects. The present invention utilizes such information in guiding the development of new therapeutic compounds, or combinations of compounds, and treatment regimens. In view of the physiological information pertaining to cocaine, treatment regimens that directly effect the activity of the dopamine and serotonin transporters, while having substantially lesser or no effect on noradrenaline transporters, are desirable.

B. Description of the Related Art

The worldwide pharmaceutical industry spends more than $30 billion a year on research and development, of which nearly one-third is spent on the discovery and early development phase, which is the period leading up to the selection of a drug candidate for preclinical and clinical development. Some critical steps in drug discovery include (1) sequencing DNA comprising segments of the human genome; (2) identification of genes within the genome that are associated with specific diseases or biological functions; (3) production of a protein such as a receptor or enzyme that corresponds to, or is encoded by, the functional gene and which then becomes a biological or molecular target for drug discovery; (4) screening a library of chemical compounds for activity against the molecular target (high throughput screening); (5) screening the most potent active compounds against other biological targets (particularly other receptors or enzymes) to assess the compounds' selectivity or specificity for the intended biological/molecular target and potential to cause undesirable side effects through activity at other targets; (6) evaluating the most potent and selective compounds for their activity in a range of other assays designed to measure such properties as toxicity, absorption, distribution, metabolism, excretion, etc.; (7) assessing the most promising compounds based on empirical judgments using the above information, and then sending that information to a chemical synthesis group to produce analogs (or modified but related chemical structures) of the initial active compounds; (8) retesting the chemical analogs through Steps (4), (5) and (6), then repeating Step (7) until an optimized lead compound or series of compounds is identified; and (9) forwarding the optimized lead compounds to further preclinical and clinical testing.

Throughout this process of discovery and development, compounds go through successively narrower filters, and compounds are eventually selected for the more expensive phases of preclinical and clinical development. Unfortunately, the selection process often leads to preclinical testing and clinical testing of compounds that will fail at these stages and never reach commercialization. These failures lead to extremely high average costs, estimated to exceed $300 million, to develop and launch a new drug. If, however, the optimal drug candidate is correctly identified early in the discovery and development process and successfully passes preclinical and clinical testing, the actual cost to develop that drug may be reduced by as much as 75%. Clearly, a major goal of pharmaceutical R&D should be to enhance the predictability of early drug development tests such as outlined above.

With the revolution of new techniques in biotechnology and the evolution of tools to automate many laboratory processes, two dominant trends have emerged in recent years that are having an important impact on pharmaceutical R&D. First, the number of molecular targets (such as new receptors and enzymes) available for discovery screening programs continues to increase dramatically due to progress in sequencing the human genome. About 500 molecular targets have been explored for drug discovery; estimates of the number of potential molecular targets that may be elucidated from the human genome project range in the thousands to more than 10,000. Second, the size of chemical compound libraries available for discovery screening programs has expanded nearly ten-fold (to more than a million compounds in many drug companies) due to automation and new technologies such as combinatorial chemistry. These two factors hold tremendous promise for new drug discovery, but they also create significant potential problems having adverse consequences on the cost of drug development. More targets and more compounds will result in many more bioactive compounds being discovered, leading to greater difficulty in selecting the optimal drug candidates to advance to preclinical testing, as well as increased development costs due to more compounds entering preclinical and clinical testing and potentially more failures at these stages.

These factors point to an increased need for rapid, inexpensive, in vitro (“test-tube” or microplate-based) assays for lead compound selection, optimization, and validation. Such rapid assays may help identify the most promising of these active compounds before they enter the later more expensive stages of drug development. These factors further point to a need for more effective methods to manage and interpret the vast amount of data on genes and gene products (molecular targets), chemical structures, and screening results.

One application of in vitro assays that is gaining increased importance in pharmaceutical R&D is “profiling.” The Assignee of this patent application pioneered the concept of profiling in the late 1980's. Drug companies are provided with an extraordinarily broad array of in vitro assays for characterizing the pharmaceutical activity and the potential side effects of compounds under development as new drugs. Currently there are more than 300 different assays that may be performed on a routine basis based on molecular targets, called receptors and enzymes, that play a key role in a wide range of human diseases, including those associated with central nervous system disorders, immune diseases, pain and inflammation, infectious diseases, cancer, metabolism or growth factors, cardiovascular function, and the endocrine system. Pharmaceuticals accounting for more than one-half of the worldwide market function by interacting with cellular receptors. In addition, many side effects of pharmaceuticals are also mediated through their interactions with receptors or enzymes.

Through profiling, a drug company's lead compounds, generally those entering preclinical development, are tested in a battery of receptor and enzyme assays. Information from the profiling process about interactions between the drug company's compound and certain receptors are important for the process of lead compound optimization and selection and can suggest possible side effects or secondary therapeutic activities of the compound. This knowledge can potentially save the drug company millions of dollars in wasted time and expense during preclinical and/or clinical development of the compound.

While profiling services have been practiced for many years, the data generated from these tests are generally used empirically by drug companies. Most drugs, even highly selective drugs, interact with numerous receptors or other molecular targets. Interpreting data produced by profiling, therefore, depends on the experience and knowledge of the scientist from the drug company who reviews the data on both the chemical structure of the compounds and the binding interactions of the compounds with specific receptors. Unfortunately, even the most experienced pharmacologist has an incomplete knowledge of the interaction of different drug compounds with the broad range of receptors relevant to drug development.

The need for more effective methods to manage, collate, interpret, and utilize the vast amount of data on genes and gene products (molecular targets), chemical structures, and screening results has led to the creation of new opportunities in bioinformatics and chemoinformatics, or managing biological and chemical data. The stages of generating large pools of information for drug discovery can be broken down into (1) DNA sequences (code of genetic material or genes that are blueprints for the cell to make gene products or proteins); (2) functional genomics process of conversion of DNA sequences to expression of corresponding gene products or proteins via mRNA production, especially in response to drugs or changes in biological function); (3) proteomics (identification of the amino acid sequence and/or three-dimensional structure of gene products or proteins, such as receptors, for which the genes code); (4) small molecule pharmacology/toxicology (molecular binding or interactions between gene products, like receptors, and small organic chemicals that are potential drugs); and (5) chemical structure (of small molecule, drug-like compounds).

Databases for DNA sequences (Group 1) are well established and include GenBank, The Genome Center, and others. Similarly, databases of chemical structures (Group 5) are well known and provided by vendors such as MDL (Isis) and Oxford Molecular. Databases for proteomics (Group 3), such as SWISS-PROT, ProLink, and PDB, are also being established. Each of these databases can be considered as one-component, in that they contain structural information and can be used to determine patterns in that one dimension or single component of structural or sequence information. Databases for Groups 2 and 4 are not well established but should be valuable additions to the information pool for drug discovery and development. These latter two forms of datasets would be two-component or two-dimensional in that they would contain data relating to the interaction between two structures, such as genes to proteins (Group 2) and proteins to chemicals (Group 4). Such relationship databases add a significant level of complexity compared with the one-component databases.

Partial databases or datasets for Group 4 relationships have been or are being established. For example, profiles of the binding of single compounds against a broad set of receptor targets by the Assignee for its clients is a partial dataset for Group 4-type databases. Similarly, data generated through high throughput screening projects in which thousands to hundreds of thousands of chemicals, such as might be contained in a chemical structure database (Group 5), are screened for activity against a specific receptor target (a single point in a Group 3 database), would represent a partial Group 4 database. Although such partial Group 4 datasets will be helpful aids for drug discovery and development, they suffer from two major drawbacks. First, they are directed toward specific two-component analyses, such as the binding selectivity of a single compound or limited set of compounds across a range of receptors (profile) or of many compounds at one receptor target (high throughput screening). In both cases, the breadth of the dataset is insufficient to allow statistical correlations to be drawn among a multiplicity of receptor targets and a multiplicity of chemical structures. Second, and importantly, these partial datasets are being generated on chemical compounds selected for their structural novelty and therefore proprietary potential as new drugs. Since these are novel compounds, there does not exist any biological information about the activity of these compounds in animals or humans. Such approaches therefore suffer the same limitations as the pharmacologist trying to empirically interpret the data of a profile, as described above.

One application for the datasets described above is in the broad area of drug discovery and development pertaining, in particular, to the development of new treatment regimens for treating drug addiction and abuse and related diseases.

Scientists have learned much about the biochemical processes involved in the human brain related to such basic behaviors as pleasure, reward, excitement, fear, anxiety, sleep, etc. Central to these phenomena are the release from nerve cells, the extracellular activity, and the reuptake back into nerve cells of a group of neurotransmitter chemicals called catecholamines, which include dopamine, serotonin, and norepinephrine. The extracellular activity of these chemicals is primarily mediated by binding of the neurotransmitters to cell surface receptors, and the reuptake is accomplished by transporters that bridge through the cell membrane. Receptors for the neurotransmitters exist in numerous forms, or subtypes, and are distributed in different tissues and organs in the body.

Substances that make humans feel good all have a remarkably similar effect on a region of the brain called the “pleasure” or “reward” center. Nearly all of these substances have the capacity to increase the levels of dopamine in the nerve synapses in the “pleasure” center of the brain. Some substances have a direct effect on dopamine, others have an apparent indirect effect mediated by interactions between the substances and other types of receptors and transporters. The end result is the same, however. The feeling of pleasure resulting from the heightened levels of dopamine can lead to the behavior of “reward” by continuing to feed the brain with the pleasure-inducing substance to maintain the high dopamine levels. This is the essence of addiction. There are numerous substances, or chemicals that are components of natural materials, that are subject to abuse and that on repeated use can become addictive. Dependency on such chemicals can have severe adverse psychological, societal, and economic impacts. The pleasure inducing substance can be cocaine, heroin, amphetamines (speed), nicotine, alcohol, barbiturates, marijuana, or any number of other drugs of abuse, or they can be pharmaceuticals intended to have other beneficial effects, or they can even be genetic, environmental, or behavioral factors themselves. So there are also numerous pharmaceuticals that, while performing a positive purpose as denoted by their therapeutic indication approved by regulatory authorities such as the Food and Drug Administration, can themselves become addictive on repeated dosing and may become abused.

While the end result is basically the same, the means is different. Blocking drug addiction for specific substances therefore requires an understanding of the complex mechanisms and interactions leading up to the elevated dopamine levels. Furthermore, since the perturbations associated with addiction are associated with effects common to a wide range of emotional or behavioral factors associated with numerous central nervous system diseases, understanding this complex set of targets can form the basis of finding improved drugs for treating diseases other than drug addiction, such as depression, attention deficit hyperactivity disorder, obesity or other eating or compulsive disorders, anxiety, etc., that also represent enormous potential markets and commercial opportunities.

One goal of pharmaceutical research and development is to discover and develop compounds or treatment regimens to combat drug addiction and dependency. One approach toward this goal has been to identify functional antagonists to the abused substance. An area in which this approach has been successfully employed is in the development and use of methadone to treat heroin addiction. Often, however, attempts to develop such treatment regimens have been hampered by a lack of understanding of the complex set of interactions between addictive substances and the molecular targets by which they exert their direct influence.

One example of a widely abused substance is cocaine. Cocaine addiction is a serious social issue. Cocaine dependency and abuse have become an epidemic, impacting the lives of many in our society. Numerous studies indicate that cocaine-associated crime now costs more than 50 billion dollars in the U.S. per year. The death rate, both by overdose and related criminal activity, is significant. Yet, there is presently no successful treatment available for cocaine addiction. Past endeavors aimed at treating such abuse with drugs have not met with success. Indeed, when compared with treating other substances of abuse, an effective treatment for cocaine dependency has eluded medical research. The need for such a treatment is clearly urgent.

Treating cocaine addiction has been a scientific challenge for a number of reasons. The lack of success in treating such insidious addiction and dependency has traditionally been attributed to the “promiscuity” of cocaine towards an assortment of central nervous system-related receptors, ion channels and transporters. The conventional wisdom (Marsh, 1998; Methews, 1983; and Smith, 1999) was that unlike other addictive drugs or chemical substances, cocaine acts on a multitude of these central nervous system related molecular targets, thus producing an intricate and complex web of neurological, physiological, and psychological effects. According to extensive reports in the past literature (Mash, 1998; Herz, 1998, Chait, 1987; Shuster, 1991, Gorelick, 1998; Giros, 1996; Sora, 1998; Rocha, 1998; 1998; Klein, 1998; Koob, 1998; Ali, 1998; Self, 1995; 1996), cocaine interacts with the dopamine, serotonin or noradrenaline transporters, with many of the dopaminergic, serotoninergic and adrenergic receptor subtypes, with the opioid, muscarinic, cholinergic and sigma-receptor subtypes, and also with many sodium and calcium channel subtypes.

This extensive literature has proven to be a labyrinth for those attempting to treat cocaine addiction based on disruption of a specific cocaine-molecular target (either receptor, ion channel, transporter and enzyme) interaction by chemical interventions and or therapeutic replacements. For instance, a typical approach of finding the “magic bullet” toward any given receptor subtype, such as dopamine (DA) receptor subtype selective ligands, has thus far proven ineffective. Dopamine 1 (D1) or D3 receptors are reportedly involved in cocaine's activity, however, their specific roles are uncertain. For example, “the use of DA receptor knockout mice has revealed a cocaine-conditioned place reference even in mice lacking the DA receptor . . . [that] suggests the possibility that other mechanisms are involved in the reinforcement caused by the cocaine administration” was noted in the recent review article by Smith et al. (Smith, 1999 and references therein). Likewise, many such attempts, either targeting other dopamine receptor subtypes or other individual molecular targets, result in phenomenology that is mired in the same, almost overwhelming, complexity and the demonstration of lack of efficacy.

Currently, addiction to cocaine is often reportedly thought to be associated with the effective blocking of dopamine transporters. Although it is known that cocaine also blocks other families of molecular targets, the selection and design of cocaine therapeutic regimens has remained primarily focused on the dopamine system. Traditionally, treatment of a particular disease or illness is based on the so-called “magic bullet” approach to discover or identify new drugs, relying on the hypothesis of the so-called “lock and key” mechanism. Essentially, if one can define an individual biological target, regardless of the nature of that target (e.g., enzyme, receptor, ion channel etc.), that is critical in the cause of the underlying illness, then the goal of drug discovery is to find a chemical entity (the key) that is specifically reactive with the target (the lock). This is the primary premise of the entire drug discovery approach. Essentially, traditional drug discovery is guided by the recognition of single pair of key/lock molecular interactions. In the case of cocaine addiction, such an approach has not been successful.

### SUMMARY OF THE INVENTION

Accordingly, it is an object of the present invention to meet the foregoing needs by providing systems and methods for analyzing data relevant to drug discovery and development. A full-rank screening database including positive and negative data resulting from a large number of chemical compounds tested against a large number of molecular targets is provided. The number of combinations of chemical compounds and molecular targets must be large enough such that a person of ordinary skill in the art of statistical or other data mining methods can use the screening database together with the corresponding chemical compound database and molecular target database to produce a reliable prediction of which chemical compounds are suitable for clinical testing and have an enhanced probability to be safe and effective drugs.

Specifically, systems and methods for meeting the foregoing needs are disclosed. The system includes a computer system comprising a first database containing records corresponding to a plurality of chemical compounds and records corresponding to biological information related to effects of the plurality of chemical compounds on biological systems of humans or animals, and a second database containing records corresponding to a plurality of molecular targets. The computer system further comprises a third database containing records corresponding to tests of binding, reactivity, or other interactions between compounds in the first database and molecular targets in the second database, the tests including information on the effect that a compound from the plurality of compounds in the first database has on the interaction between a selected compound (e.g., a reference agent or standard) known to interact with a specific molecular target from among the plurality of molecular targets, said tests being performed for a plurality of the molecular targets in the second database. Means for setting an interaction test threshold corresponding to said effect and means for selecting the compound, sets of compounds, and/or information associated with such compound(s) when the results of the testing of the effect meet the interaction test threshold are also included in the computer system. A user interface is provided to allow a user to view and manipulate or analyze information from the first database, the second database, and the third database as it relates to one or more compound records in the first database and/or as it relates to one or more molecular target records in the second database, especially with respect to compounds, molecular targets, or other database records associated with results that meet the interaction test threshold(s). Furthermore, the invention relates to using methods of statistical analysis and other data mining methods as applied to these multidimensional databases to determine correlations or patterns that are relevant to drug discovery and development.

It is another object of the present invention to provide a systematic method for identifying the causal relationships of an abused chemical or substance and physiologically relevant molecular target sets. The method is useful in identifying relationships between many types of addictive chemicals or substances and molecular target sets, leading to chemical therapeutic interventions.

In another broad aspect, the invention relates to a method for determining a biological activity profile for an abused or addictive substance, comprising:

(a) selecting a panel of molecular targets;

(b) defining a pharmacological activity profile for the abused substance by;

(i) exposing the abused substance to each of the molecular targets in the panel; and

(ii) measuring the ability of the abused or addictive substance to interact with the molecular targets; and

(c) determining the biological activity profile by identifying in the pharmacological activity profile a subset of the molecular targets whose activity is affected by the abused substance.

In yet another aspect, the invention relates to a method for determining a set of one or more molecular targets for developing a treatment for abuse of, or addiction to, a substance, comprising:

(a) determining a biological activity profile of an abused or addictive substance; and

(b) defining the set of molecular targets by identifying those targets in the biological activity profile wherein the interaction between the abused or addictive substance and the molecular target exceeds a threshold level.

In a particular embodiment, the method of the invention further comprises utilizing information relating the pathology associated with abuse or addiction to the substance to interaction of the substance with different molecular targets to define the set of molecular targets. In a specific embodiment, the information utilized includes either or both of information concerning at least one positive effect of the abused or addictive substance and information concerning at least one negative effect of the abused or addictive substance. In certain embodiments, the information exists in a relational database.

Another broad aspect of the invention relates to a method for identifying at least one chemical compound to treat abuse or addiction of a substance, comprising:

(a) determining a set of molecular targets;

(b) providing a database of chemical compounds containing records corresponding to a plurality of chemical compounds, wherein said records include data on the interaction of the chemical compounds with a plurality of molecular targets; and

(c) selecting one or more chemical compounds from the database, wherein the selected chemical compound or compounds interact with the molecular targets in a manner substantially the same as the abused or addictive substance.

In a particular embodiment, the method of the invention further comprises utilizing information relating the pathology associated with abuse or addiction to the substance to interaction of the substance with different molecular targets to define the set of molecular targets. In a particular embodiment, the information exists in a relational database.

In certain embodiments, the pharmacological activity profile is determined by the use of one or more assays. Each assay comprises a buffer solution, a cell or tissue preparation containing a molecular target, and a labeled compound that interacts with the molecular target. In alternative embodiments, the molecular target is a receptor, transporter, ion channel, or enzyme. The molecular target may be from animal tissue, human tissue, or cultured cells. The cultured cells may express a native molecular target or, in an alternative embodiment express a recombinant nucleic acid encoding the molecular target. In certain embodiments, the molecular target is a crude preparation, partially purified, or highly purified.

In the assays, the labeled compound is a small organic molecule, a peptide, a nucleic acid, an oligosaccharide, or a macromolecule. In certain embodiments, the macromolecule is a protein, polysaccharide, DNA, or RNA. In particular embodiments, the compound is labeled with a radioisotope, a fluorescent tag, a bioluminescent tag, or a chemoluminescent tag. Specifically, the radioisotope is ³H, ¹⁴C, ¹²⁵I, or ³²P.

Alternatively, the labeled compound is a substrate for an enzyme and the one or more of the assays is an enzyme assay wherein the substrate has a measurable characteristic. In some embodiments, the measurable characteristic is UV or visible absorbance or fluorescence.

In some embodiments, the one or more assays is a functional assay. In other embodiments, the labeled compound interacts with the molecular target, is a substrate for an enzymatic reaction, or alters the function of an ion channel or transporter. In certain embodiments, the compound binds to the molecular target and induces a detectable signal. In particular embodiments, the detectable signal is a chemoluminescent output, a bioluminescent output, a morphological change, or a colorimetric change.

In other embodiments, the assay detects a secondary signal. In alternative embodiments, the secondary signal is cAMP, Ca2+ flux, membrane depolarization, IP3 turnover, neurotransmitter release, or ion transport.

In another embodiment, the invention is directed to a method of treating substance abuse, comprising:

(a) determining a set of molecular targets;

(b) providing a database of chemical compounds containing records corresponding to a plurality of chemical compounds, wherein said records include data on the interaction of the chemical compounds with a plurality of molecular targets;

(c) selecting one or more chemical compounds from the database, wherein the selected chemical compound or compounds interact with the molecular targets in a manner substantially the same as the abused or addictive substance; and

(d) administering to a patient in need thereof an effective amount of at least one of the selected chemical compounds.

Another embodiment of the invention is a computer system comprising:

(a) a database containing records corresponding to a plurality of addictive substances, wherein said records include chemoinformatic information, in vivo biochemical information, and information concerning the physiological effects of the addictive substance; and

(b) a user interface allowing a user to view records of the addictive substances.

In a particular embodiment, the invention is related to a method for determining a biological activity profile for cocaine, comprising:

(a) selecting a panel of molecular targets;

(b) defining a pharmacological activity profile for cocaine by;

(i) exposing cocaine to each of the molecular targets in the panel; and

(ii) measuring the ability of cocaine to interact with the molecular targets; and

(c) determining the biological activity profile by identifying in the pharmacological activity profile a subset of the molecular targets whose activity is affected by cocaine.

Another specific embodiment of the invention is a method for determining a set of one or more molecular targets for developing a treatment for cocaine addiction, comprising:

(a) determining a biological activity profile for cocaine; and

(b) defining the set of molecular targets by identifying those targets in the biological activity profile wherein the interaction between cocaine and the molecular target exceeds a threshold level.

In a related embodiment the invention further comprises utilizing information relating the pathology associated with cocaine addiction to the interaction of cocaine with different molecular targets to define the set of molecular targets. In certain embodiments, the information exists in a relational database.

Another embodiment of the invention is a method for identifying at least one chemical compound to treat cocaine addiction, comprising:

(a) determining a set of molecular targets for developing a treatment for cocaine addiction;

(b) providing a database of chemical compounds containing records corresponding to a plurality of chemical compounds, wherein said records include data on the interaction of the chemical compounds with a plurality of molecular targets; and

(c) selecting one or more chemical compounds from the database, wherein the selected chemical compound or compounds interact with the molecular targets in a manner substantially the same as cocaine.

In one embodiment, the invention relates to a method of treating cocaine addiction, comprising:

(a) determining a set of molecular targets for developing a treatment for cocaine addiction;

(b) providing a database of chemical compounds containing records corresponding to a plurality of chemical compounds, wherein said records include data on the interaction of the chemical compounds with a plurality of molecular targets;

(c) selecting one or more chemical compounds from the database, wherein the selected chemical compound or compounds interact with the molecular targets in a manner substantially the same as cocaine; and

(d) administering to a patient in need thereof an effective amount of at least one of the selected chemical compounds.

In a specific embodiment, the invention is directed to a method of treating cocaine addiction, comprising administering to a patient in need thereof an effective amount of a pharmaceutical composition comprising at least one compound that directly effects the activity of the dopamine and serotonin transporters and has substantially no effect on noradrenaline transporters.

These and any other embodiments of the invention, which are described in more detail below, provide methods for identifying sets of biological targets that are useful in guiding the selection of therapeutics to treat substance abuse, addiction and dependency.

Both the foregoing summary of the invention and the following detailed description provide examples and explanations only. They do not restrict the claimed invention.

### BRIEF DESCRIPTION OF THE DRAWINGS

The accompanying drawings, which are incorporated in and constitute a part of this specification, illustrate embodiments of the invention and, together with the description, explain the advantages and principles of the invention.

FIG. 1A illustrates a chemical compound table in the receptor selectivity mapping database according to one embodiment of the present invention;

FIG. 1B illustrates a snap-shot of a chemical compound record containing spatial coordinates of a compound in the receptor selectivity mapping database according to one embodiment of the present invention;

FIG. 2 illustrates several logical tables that may be used to access the molecular target information in the receptor selectivity mapping database according to one embodiment of the present invention;

FIG. 3 illustrates a biological information table in the receptor selectivity mapping database according to one embodiment of the present invention;

FIG. 4 illustrates the use of a receptor selectivity mapping database as part of a screening process according to one embodiment of the present invention; and

FIG. 5A illustrates the use of a receptor selectivity mapping database as part of a screening process to discover and select new compounds as potential new drug candidates for further development.

FIG. 5B illustrates the use of a receptor selectivity database as part of a screening process to identify new targets as potential validated targets to use to discover new drug candidates for specific disease indications.

FIG. 6A illustrates the use of a database for predicting the drug potential of a new compound.

FIG. 6B illustrates the use of a database to validate the disease relevance and/or the biological function of a new molecular target.

FIG. 7 illustrates a general strategy of identifying molecular target sets useful in designing regimens to treat chemical dependency and addictions.

FIG. 8 provides an example of using an in vitro biological activity profile of chemicals and a database comprised of information of chemical and biological or molecular target-based physiology to design a set of molecular targets to use as guide a for the selection and design of treatment regimens for cocaine addiction and dependency.

FIG. 9 shows a database screenshot containing in vitro biological activity data (Pass 1 and Pass 2) determined as a result of testing a chemical compound (cocaine) for activity at molecular targets.

FIG. 10 shows a database screenshot containing in vitro biological activity data (Pass 3 and quantitative potency, or IC₅₀/Kᵢ values) determined as a result of testing a chemical compound (cocaine) for activity at molecular targets.

FIG. 11 shows a database screenshot containing chemoinformatic annotations (chemical information) for a chemical compound (cocaine) in the system database.

FIG. 12 shows a database screenshot containing physiological annotations (in vivo activities and effects) for a chemical compound (cocaine) in the system database.

FIG. 13 shows a database screenshot containing toxicological annotations (in vivo activities and effects) for a chemical compound (cocaine) in the system database.

FIG. 14 shows a database screenshot containing bioinformatic annotations (biochemical and structure properties and information) for a molecular target (serotonin transporter) in the system database.

FIG. 15 shows a database screenshot demonstrating linked tables in the database between bioinformatic annotations, chemical reactivity profiles, and potencies of interactions for chemical compounds (cocaine) tested against a molecular target (serotonin transporter).

FIG. 16 shows a screenshot of an intranet web page comprising a portion of an interface to the system database used to interrogate information housed in the database, including the demonstration of switches to select and compare different components of the database.

FIG. 17 shows a screenshot of an intranet web page comprising a portion of an interface to the system database used to select various threshold ranges for biological activity.

### DETAILED DESCRIPTION OF THE INVENTION

Reference will now be made to preferred embodiments of this invention, examples of which are shown in the accompanying drawings and will be obvious from the description of the invention. In the drawings, the same reference numbers represent the same or similar elements in the different drawings whenever possible.

Systems and methods consistent with the present invention allow the analysis of data relevant to drug discovery and development and the prediction of the potential of a new compound, for example, for its suitability for progression to preclinical and clinical tests with an enhanced probability of becoming a safe or effective new drug. For purposes of the following description, the systems and methods consistent with the present invention are described with respect to a relational database containing multiple main tables and with the use of the binding between chemical compounds and molecular targets as a measurement of the interactions between the two. The description should also be understood to apply in general for any database structure having multiple main components and to the measurement of any interactions between chemical compounds and molecular targets.

The present invention relates to the novel design, construction, and application of a database relating information-rich chemicals, molecular targets, especially proteins or other macromolecules, and biological activity of the chemicals. Furthermore, the present invention relates to the primary use of known drugs and drug candidates that have failed in clinical or preclinical trials as a source of the chemical library for the database, together with preclinical or clinical data generated for such chemicals describing their side effects, mechanism of action and other medically relevant data. The present invention further relates to determining the binding or other interactions between the chemicals and the molecular targets in the database, then using methods of relationship analysis and data mining to correlate patterns of these interactions with specific biological activities that are relevant to drug discovery and development, or with specific chemical structures, substructures, or other features of compounds exhibiting such interactions, or with biochemical, structural, or other features of molecular targets exhibiting such interactions. Examples of such data mining techniques can be found in the following references, which are incorporated by reference in their entirety:

a) Chen et al., Recursive Partitioning Analysis of a Large Structure-Activity Data Set Using Three-Dimensional Descriptors, Journal of Chemical Information and Computer Sciences, October 1998;

b) Hawkins et al., Analysis of a Large Structure-Activity Data Set Using Recursive Partitioning, Quant. Struct.-Act. Relat., 16:296-302 (1997);

c) DePriest et al., 3D-QSAR of angiotensin-converting enzyme and thermolysin inhibitors; a comparison of CoMFA models based on deduced and experimentally determined active site geometries, J. Am. Chem. Soc., 115:5372-84 (1993);

d) Good et al., in Reviews in Computational Chemistry; Lipkowitz, K. B., Boyd, D. B. (eds.), VCH, New York, Vol. 7, pp 67-117 (1996);

e) Marshal et al., in Computer-Assessed Drug Design; ACS Symposium Scrica 112; American Chemical Society: Washington, D.C., 1979; pp 205-226;

f) Moloc et al., A three-dimensional structure activity relationships and biological receptor mapping, in Mathematics and Computational Concepts in Chemistry; Ellis Horwood; Chichester, 1985; pp 225-251;

g) Mayer et al., A unique geometry of the active site of angiotensin-converting-enzyme consistent with structure activity studies, J. Comput. Aided Mol. Des., 1:3-16. (1987);

h) Sheridan et al., The ensemble approach to distance geometry: application to the nicotinic pharmacophone, J. Med Chem. 29:899-906 (1986);

i) Martin et al., A fast new approach to pharmacophone mapping and its application to dopaminergic and benzodiazepine agonists, J. Comput. Aided Mol. Des., 7;83-102 (1993);

j) Catalyst/Hypo Tutorial, version 2.0, BioCAD Corp. Mountain View, Calif., 1993

k) Sprague, P. W., Automated chemical hypothesis generation and database searching with Catalyst, Perspect. Drug Discov. Des., 3:1-20 (1995);

l) Barnum et al. Identification of common functional configurations among molecules, J. Chem. Inf. Comput. Sci., 1996, 36:563-71 (1996).

m) HipHop Tutorial, version 2.3; Molecular Simulation Inc.; Sunnyvale, Calif., 1995;

n) Davies, K. and Upinn, R., 3D pharmacophore searching, Net. Sci., (www.netsci.org/Science/Cheminform/feature02.html);

o) Golender, V. and Vesterman, B., APEX 3D expert system for drug design, Net. Sci. (www.awod.com/netsci/Science/Compchem/feature09.html);

p) Van Drie, J., Strategies for the determination of pharmacophoric 3D database queries, J. Comput. Aided Mol. Des., 11:39-52 (1997);

q) Van Drie, J. and Nugent, R., Addressing the challenges posed by combination chemistry: 3D databases, pharmacophon; recognition and beyond, SAR QSAR Environ. Res., 9:1-21 (1998);

r) Finn et al., Pharmacophore discovery using the inductive logic programming progol, in Machine Learning, Special Issue on Applications and Knowledge Discovery, Kluwer Academic Publishers: Boston, 1998, pp 1-33; and

s) Jain et al., Compass: a shape-based machine learning tool for drug design. J. Comput. Aided Mol. Des., 8:635-52 (1994).

The background section suggests that, contrary to standard operating procedures in the pharmaceutical industry, a Group 4 database should be established having more components than a two-component database, and that it should cover a substantial breadth of both receptor or enzyme targets and chemical compounds. By way of example, a three-component database would be created by first selecting a broad set of chemical compounds that are rich in information of direct relevance to drug discovery and development. The most relevant information is often obtained by actual experience of testing such chemical compounds in humans through clinical trials and/or post-marketing surveillance or in animals through preclinical testing. Other relevant biological information may come from natural products that demonstrate one or more observed bioactivities, as well as chemical reference standards that have been used in the industry to characterize the biology of receptors. Accordingly, one embodiment of information-rich chemical compounds selected for such a Group 4 database includes marketed pharmaceuticals, drugs that have failed in clinical or preclinical trials, bioactive natural products or natural extracts, and reference agents used for receptor binding assays.

One may construct such a database using screening data obtained from the scientific literature. While this approach could yield partial datasets, it may have limitations. First, literature references generally provide only positive information (e.g., reports of inhibition of binding of a specific compound to a specific receptor) and not negative data (e.g., a lack of inhibition of binding and therefore lack of activity). In determining useful comparisons of information, negative data can be as valuable as positive data. Furthermore, certain statistical analyses may not be applicable to datasets that lack completeness of both positive and negative data. Second, separate quantitative reports of binding data for one compound against a receptor in one article vs. reports of binding data for a second compound at the same receptor may not be comparable because of variations in the way the assays were performed. Therefore, one embodiment for creation of a Group 4 three-component database would be to screen a broad array of compounds through a broad array of receptor or enzyme targets in order to obtain consistent comparative results and ensure the collection of both positive and negative data.

The Chemical Compound Component: Selection of Chemical Libraries and Inclusion of Chemical Data

The present invention relates to databases that contain, as one component, chemical compounds about which information is known concerning biological activity relevant to pharmaceutical research and development. The biological activity information may be included in the chemical compound database or table.

These information-rich chemicals include:

(a) Compounds that are pharmacological reference agents or reference standards for measuring the interaction or molecular binding between unknown chemical compounds and a specific molecular target, such as a receptor or enzyme. Examples of such reference compounds include those compounds that are used for characterizing binding interactions between test compounds and molecular targets including receptors or enzymes. Other reference agents could include chemicals selected from the catalog of Research Biochemicals Inc. (RBI), a unit of Sigma Aldrich Corp., and from other sources that are well known in the industry. These pharmacological reference compounds often have been tested previously and/or marketed as pharmaceuticals or are natural products with characterized biological activity and therefore may overlap with compounds in the following three categories;

(b) Compounds that are known pharmaceuticals that are currently or have previously been marketed for clinical use, and for which there is a substantial amount of biological information available. These compounds are well-known and are listed in publications available from U.S. government agencies such as the Food and Drug Administration (FDA), as well as publications by private or non-profit organizations. One such publication by a non-profit organization is the United States Pharmacopeial Convention Inc.'s USP DI Series, including Volume I. Drug Information for the Health Care Professional, which is updated monthly by USP DI Update. As new drugs are approved for marketing, they would be included in this category. Marketed pharmaceuticals or drugs approved by the FDA or equivalent foreign regulatory bodies are a matter of public record so that one normally skilled in the art can easily identify chemical compounds that would be included in this category;

(c) Compounds that have been approved for testing in humans, such as compounds that had been granted IND (Investigational New Drug) status, as potential drugs but that failed to achieve sufficient efficacy or safety in clinical trials to gain approval from the FDA or otherwise did not reach the status of marketed pharmaceuticals. Compounds in this category may also include those compounds that have been approved by the FDA for commercialization but that have later been withdrawn from the market. These compounds also would have a significant amount of biological information available and would be especially useful for purposes of this invention. The identity of failed drugs can be obtained from numerous sources, including public announcements by drug and biotechnology companies, publications such as the “Pink Sheets”, and lists maintained by the Food and Drug Administration (“FDA”); and

(d) Compounds that are obtained from natural sources such as plants, microorganisms, animals, etc., that exhibit biological activity. These natural products may include toxins, antimicrobial agents, behavioral modifiers, defensive agents, and other categories of compounds that provide information relevant to drug discovery and development. The identity of natural products can be found in numerous publications, including but not limited to, the RBI catalog and Sigma Aldrich catalog of chemical compounds.

For each compound included in the database, chemical structure, chemical formulae, physical chemical characteristics, chemical space coordinates or other chemical structure descriptors (e.g., Smiles codes), solubility, and other relevant data, to the extent such information is available, are entered into fields in the database. Those skilled in the art would recognize other parameters that might be included. Chemicals can be organized by chemical structure relatedness in the database or in other relationships.

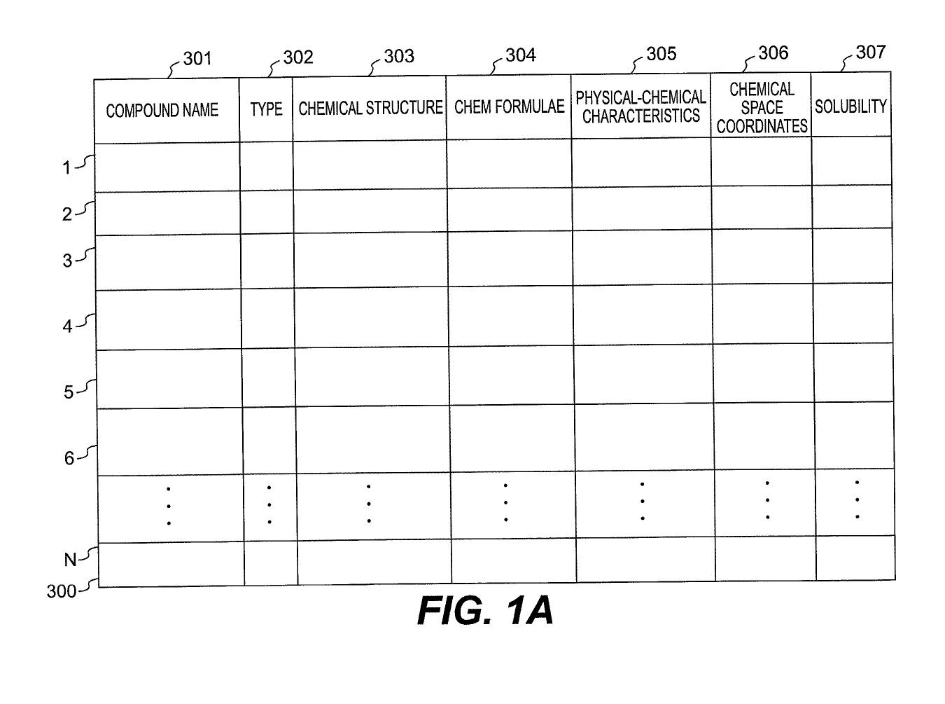

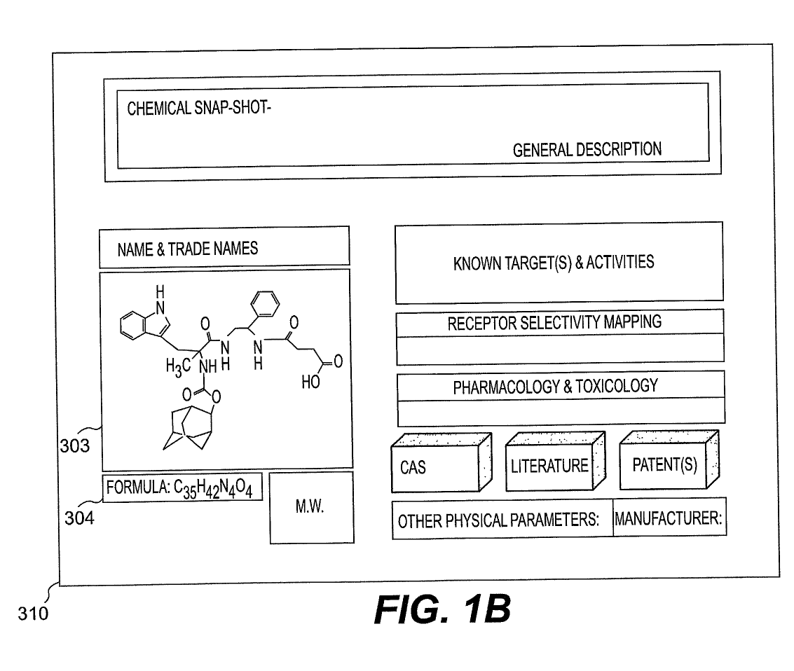

FIG. 1A illustrates a chemical compound table 300 in a relational database system. The table 300 lists a number of chemical compounds and includes records (rows 1-N) of a number of compounds N. For each compound there may be a number of corresponding columns 301-307 containing information related to the compound. For example, in FIG. 1A column 301 contains the name of the compound; column 302 includes the compound type (e.g., compounds that have been approved for testing in humans, etc.); column 303 includes information related to the chemical structure, for example, a hyperlink that brings up a screen containing a drawing of the structure (see snap-shot 310 in FIG. 1B); column 304 includes the chemical formula for the compound; column 305 includes information about the physical-chemical characteristics of the compound; column 306 includes chemical space coordinates of the compound; and column 307 includes solubility information of the compound.

Additional columns may be added in order to include other relevant data related to each chemical compound 301 listed in the table 300. These additional columns may include biological activity of the compound, rendering the chemical compound database a two component database (see also database 500).

FIG. 1B illustrates a snapshot 310 that may include information corresponding to a record in the table 300. For example, the chemical formula 304 of a compound may be included in the snapshot of the record as well as the compound's structure 303.

The Molecular Target Component: Selection of Receptors, Enzymes, and Other Molecular Targets and Inclusion of Molecular Target Data

Molecular targets such as receptors, enzymes, other proteins, nucleic acids, carbohydrates, and other macromolecules relevant to drug discovery and development, are representative of the second component of the databases comprising this invention. In one embodiment of this invention, receptors and enzymes are the principal molecular targets. Receptors mediate much of the molecular communication among cells and organs in the body. Enzymes often amplify such communications through, for example, secondary messenger systems and cell signaling pathways.

Receptors include classical families of receptors such as dopamine receptors, serotonin receptors, opiate receptors, muscarinic receptors, adrenergic receptors, adenosine receptors, etc. These receptor groups include subtypes of the receptor type (such as dopamine-1, dopamine-2, dopamine-3, dopamine-4, and dopamine-5 receptors). Certain subtype have further variations (such as dopamine 4.2, dopamine 4.4, and dopamine 4.7) or can have different forms (such as dopamine 2 short and dopamine 2 long). Splice variants of receptors can also occur, as can mutations in the genes encoding specific receptors which might lead to a subset of a population that has a receptor with slightly different binding affinity for drugs or other compounds compared with the normal receptor type. Receptors can be grouped by family, superfamily, or subfamily. Some groupings include G-Protein Coupled Receptors, 7 transmembrane receptors, nuclear receptors, etc. Receptors can be grouped by the degree of homology of the DNA sequence of their corresponding genes. Receptors can also be grouped by their amino acid sequence and related three-dimensional conformations. Receptors can be classified by their location of expression in tissues or across different cell types.

Enzymes can include proteases, carbohydrases, kinases, phosphatases, DNA-modifying enzymes, transferases, P450's, and others known to those skilled in the art.

Other receptors, receptor sources, and corresponding assays are constantly being developed to be added to the content of the database. Additional receptors and receptor assays are well known to those skilled in the art. Lists and descriptions of certain receptors relevant to drug discovery and development can be found in numerous publications known to those skilled in the art. These publications include the RBI Handbook of Receptor Classification and the IUPHAR receptor classification book. Furthermore, as new receptors and receptor subtypes are discovered, they can be added to the content of the database.

Enzymes and enzyme assays are well known to those skilled in the art. Lists and descriptions of certain receptors relevant to drug discovery and development can be found in numerous publications known to those skilled in the art.

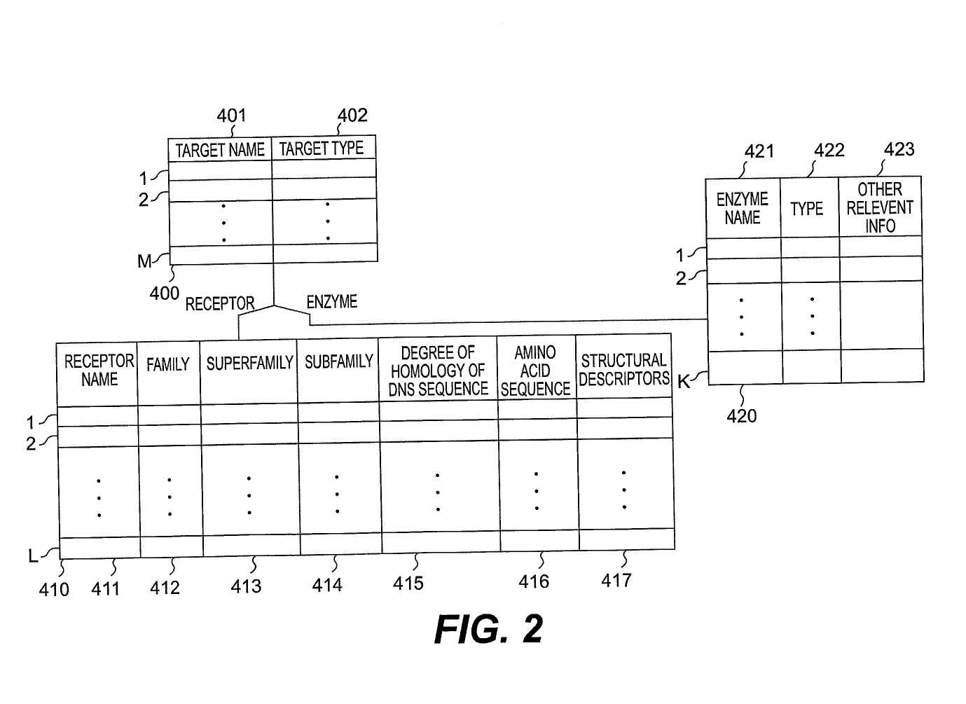

FIG. 2 illustrates tables 400, 410, and 420 forming part of a relational database system which may be used to access molecular target information. Table 400 lists the targets and includes records (rows 1-M) of a number of targets M. Column 401 lists the names of the target, while column 402 specifies the target type corresponding to each target name.

Table structures may vary according to the target type specified in column 402. Table 410 includes information about those targets listed in table 400 which are classified as receptors. Records from table 410 may be accessed by querying the database for a particular receptor name. The receptor names found in table 410 may be accessed, in turn, by querying table 400 for those target names for which column 402 reads “Receptor.”

In table 410, column 411 contains the name of the receptor, which is also the name of the target in column 401 in table 400; column 412 includes receptor family information; column 413 includes receptor superfamily information; column 414 includes receptor subfamily information; column 415 includes the information about the degree of homology of the DNA sequence of corresponding genes; and column 416 includes information on amino acid sequence. The amino acid sequence is one of a number of molecular descriptors that may be included in the database. Other molecular descriptors, for example, could include hydropathy plots corresponding to the amino acid sequence. Because the molecular target database represented by tables 400, 410, and 420 includes target information and associated biological information related to the targets is included in the database (see table 600), this database may be considered a two-component database. The columns shown are illustrative of the types of information that may be included in the database and should not be constructed as limiting the invention.

Table 420 includes information about those targets in table 400 which are classified as enzymes. Records from table 420 may be accessed by querying the database for a particular enzyme name. The enzyme names found in table 420 may be accessed, in turn, by querying table 400 for those target names for which the target type column 402 reads “Enzyme.”

In table 420, column 421 contains the name of the enzyme, which is also the name of the target in column 401 of table 400 and column 422 includes enzyme type information. Column 423 is labeled as “Other relevant information” and is included in the table for purposes of illustrating that additional columns may be added to table 420 depending on other enzyme information that a user of the database might want to access, including amino acid sequence and molecular description.

Although only tables 410 and 420 are shown to describe the access of molecular target information by using the target type, additional tables may be added to the relational database system corresponding to the number of molecular target types available in the database.

The Biological Information Component: Selection of Biological/Clinical Information Parameters

Biological information forming part of the database includes material that would relate to side effects, mechanism of drug action, metabolism of a drug, toxicity, adsorption, distribution, and excretion, for example. This information is available on FDA-approved labels of marketed drugs, or from literature sources and publications for drugs that have failed clinical trials. Examples of some specific parameters are Toxicity, LD 50, LD50/ED50, Teratogenicity, Mechanism Of Toxicity, Target Organ For Toxicity, In Vitro Toxicity Battery, Induction Of Apoptosis, Bioavailability, Absorption, Blood-Brain Barrier, Oral Absorption, Mucosal Absorption, % Absorbed, Distribution, Blood Protein Bound, Half-Life, Onset Of Action, Duration Of Action, Peak Concentration In Blood, Metabolism, Major Pathway, Minor Pathway, Active Metabolites, Excretion, Primary Excretion Mode, Secondary Excretion Modes, In Vivo Effects, Therapeutic Indication, Animal Behavioral Effects, Side Effects, Primary Known Target, Other Organ/System Targets, and Known Receptor Interactions.

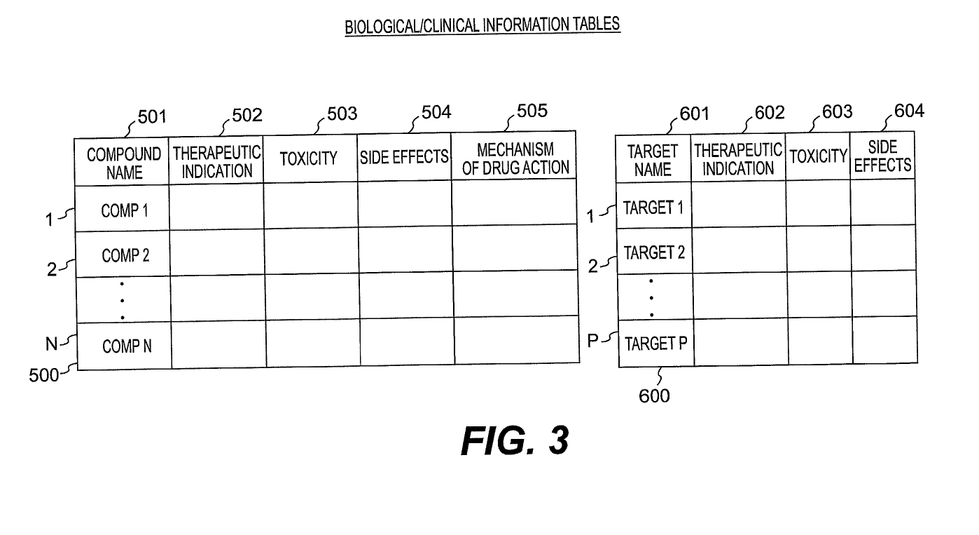

FIG. 3 shows table 500 which includes some of the biological information parameters mentioned above. Table 500 comprises N rows (1 through N) which correspond to all the possible chemical compounds in the first database. Column 501 includes the compound name; column 502 includes the therapeutic indicator (for marketed or failed drugs); column 503 includes toxicity information; column 504 includes side effects information; and column 505 includes information on the mechanism of drug action. Table 500 would be associated with table 300, for example, to form a two-component chemical compound and biological activity table.

FIG. 3 also shows Table 600, which includes biological information in parameters associated with the molecular targets in the database. Table 600 includes P rows (1 through P) which correspond to all the possible targets in the second database. Column 601 includes the target name; column 603 includes toxicity information, and column 604 includes side effects information. Similarly, table 600 would be associated with table 400, for example, to form a two-component molecular target and biological activity table. Tables 500 and 600 together may be a full-rank database (e.g., including all possible combinations between compounds and molecular targets in a relational database system) including molecular target information, chemical compound information, and biological activity information associated with each of the molecular targets and with each of the chemical compounds, and may be considered a multidimensional database. Additional columns may be included in tables 500 and 600 without departing from the invention.

Determining Binding Information

A key feature of this invention is the establishment of several components of information which, by way of illustration, comprise chemicals, molecular targets, and biological information, and measuring the binding, or reactivity or other interactions between the chemicals and molecular targets. This binding or reactivity information can then be related back to the known biological information in order to distinguish patterns and relationships that can be used for drug discovery and development. An important aspect of this invention is to generate broad and consistent binding or reactivity data between the chemicals and molecular targets in order to provide as complete a dataset as possible in order to be able to identify relevant patterns or relationships and to provide both positive and negative binding or reactivity information for the datasets. In one embodiment, the binding data is established as a numerical descriptor that either satisfies or does not satisfy a threshold set, for example, for a specific molecular target or set of molecular targets. The numerical descriptor may relate to the activity or lack of activity for each compound and each receptor or other molecular target measured at a concentration deemed near the appropriate threshold for relevance to the biological system or biological information set. For example, chemicals can be tested at 10⁻⁵ M (10 micromolar) for their ability to inhibit binding at a threshold of 30% between a receptor and its specific reference compound. Other initial concentrations or percentage inhibition thresholds can be selected. Also, in one embodiment, those chemicals that demonstrate inhibition of binding above the threshold in the initial yes/no testing are further tested for the potency of the binding inhibition. These active chemicals are tested at a series of concentrations that might, for example, include tests at 7-14 different concentrations within the range of 10⁻⁵ to 10⁻⁹ M, such that an IC-50 and/or Ki value can be determined for the active compound at the specific receptor. Fewer or more concentrations may be used for such determinations and concentrations above or below 10⁻⁵ to 10⁻⁹ M may be required. These data then yield a matrix of relative degree of activity or relative potency for each active compound at each molecular target.

In order to generate these screening data, chemicals are first solubilized in a suitable solvent system, such as 4% DMSO, although other concentrations of DMSO and other solvents are also acceptable. These chemical stock solutions are then diluted to the appropriate concentration and made available as repositories. For each assay measuring the interactions between the chemical and molecular target, the reagents and protocols for the assay will vary. Each such assay needs to be characterized and routinely established for consistency. Appropriate controls need to be run each time the assay is performed. Any assay format that can generate the desired type and accuracy of information can be used. Numerous assay detection systems, such as radioactive labels, fluorescence, fluorescence polarization, time-resolved fluorescence, fluorescence correlation spectroscopy, chemiluminescence, UV absorption, colorimetric, etc., can be used.

In one embodiment, a receptor-binding assay or enzyme activity assay is used to generate data on molecular interactions. As an example, for a receptor binding assay, chemicals from a repository are tested for their ability to inhibit the binding interaction between the receptor and a reference agent selected for that receptor. The receptor may be derived from a tissue source, such as animal or human tissue, or from a cell line expressing the receptor, or from a transfected cell line containing the gene for the receptor. The receptor source is prepared for the assays, for example by preparing a membrane fraction containing the receptor. Alternatively, the receptor may be partially purified. The reference compound, or ligand, is preferably selected for its potent and/or specific binding to the specific receptor and may have a radioactive tracer such as Iodine-125 or tritium or carbon-14 or other marker to enable a bound ligand to be distinguished from an unbound ligand. Coincident with testing the chemicals for binding data to include in the database, positive and negative controls are run, as is a reference curve with varying concentrations of the reference (radio)ligand to ensure the quality of the assay run. A plurality of methods and systems may measure the interactions between targets and compounds as would be recognized by a person of ordinary skill. The radioligand, receptor preparation, and test compounds are incubated together for an appropriate time, in an appropriate buffer, and at an appropriate temperature, often with the objective of reaching equilibrium of the binding reactions. The amount of bound vs. unbound radioligand is determined by a separation step, such as filtration, or by use of a method, such as SPA (scintillation proximity assay), and measured by liquid scintillation or gamma counting. The amount of specific binding of the test compound is then determined by comparing assay results for the test chemical(s) vs. the positive and negative controls. The percent inhibition of the test chemical(s) is calculated from these data.

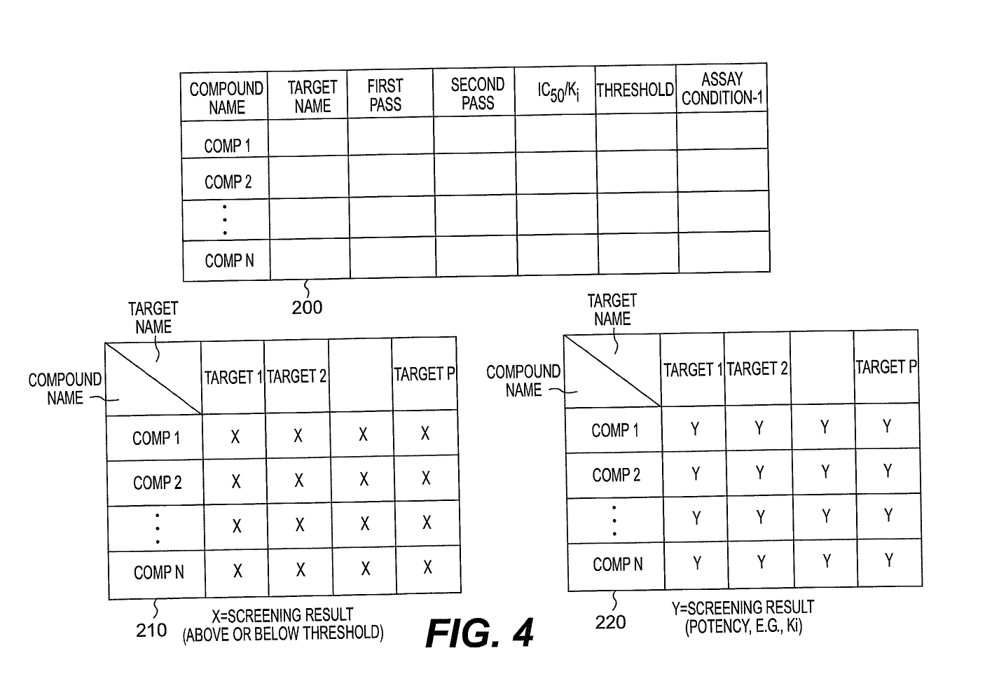

FIG. 4 shows Table 200 as an illustration of a screening results and assay database in which, for example, chemical compounds included in database 300 (comprising 1 to N chemical compounds) are tested for their effect against molecular targets included in database 400 (comprising 1 to L receptors, or alternatively, 1 to K enzymes or other included targets). Numerous forms of Table 200 are possible. For example, in Table 210 screening results are entered in a “yes” or “no” entry with respect to whether the screening result for each of a plurality of chemical compounds tested against each of a plurality of molecular targets was above or below the selected threshold test result for each set of determinations.

As another example, in Table 220 screening results are entered as a numerical descriptor identifying the potency or magnitude of the binding or other effect (e.g., the Ki for chemical:receptor interactions) for each of a plurality of chemical compounds tested against each of a plurality of molecular targets. In a preferred embodiment, all such matrix points for chemicals x targets in Tables 210 and 220 are determined and entered into the database such that a full-rank dataset is derived. The screening results and assay database 200 may also include other measurements of chemical:target interactions, including raw data of screening results and measurements derived from the raw data, assay protocols and performance characteristics, and other relevant information.

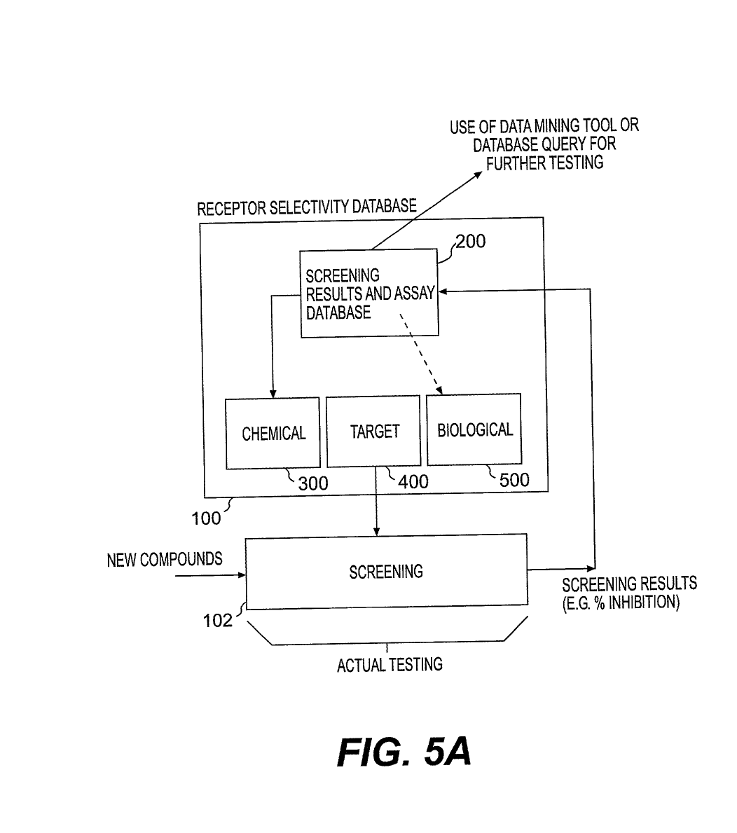

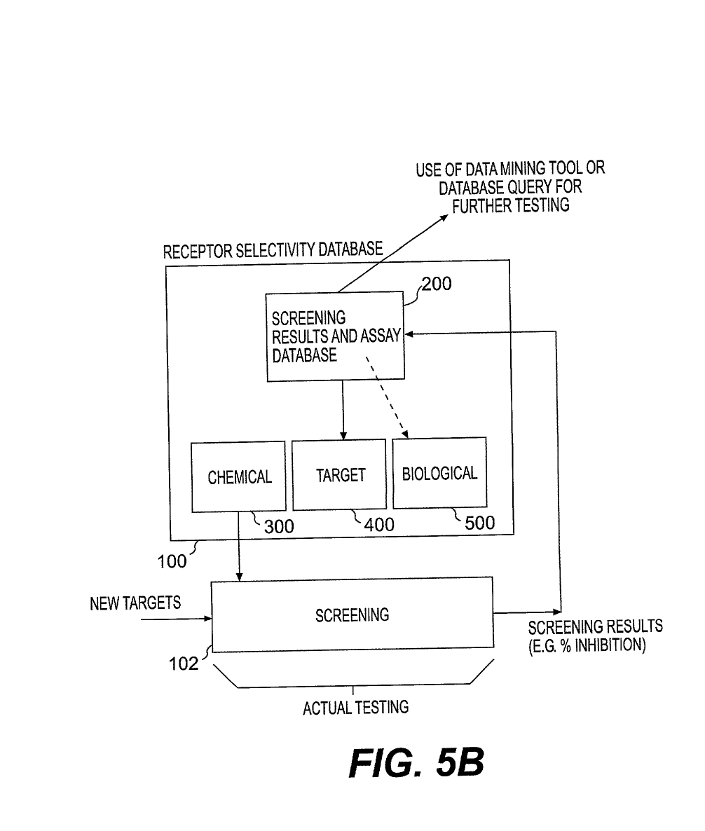

FIG. 5 illustrates the use of a database 100, here shown as a receptor selectivity database, by way of example, as part of a screening process to discover and select new compounds as potential new drug candidates for further development (FIG. 5A) or new targets as potential validated targets to use to discover new drug candidates for specific disease indications (FIG. 5B). The database 100 may include a chemical compound component 300; a molecular target component 400; biological information components 500 and 600; and a screening results and assay database 200.

A new compound or set of compounds is introduced to a screening process 102 for determining whether it is effective in inhibiting the binding of a specific chemical compound (e.g., a reference agent) and a molecular target. The screening process may use target information from the molecular target component 400.

The results of the screening process 102 may be stored in an intermediate database or continued into the screening results and assay database 200 of the receptor selectivity database 100. The results may also be stored in the biological information database 500 as particular parameters (e.g., cytotoxicity, etc.) as well as in the chemical compound database 300 (e.g. name of the compound, etc.).

The complete set of results from the screening process 102 may be stored in the screening results and assay database 200. The database 200 may be queried for those new compounds that exhibit an inhibitory effect on the binding of molecular targets and chemical compounds (e.g., reference agent) so that those new compounds can further be tested.

Alternatively, a new molecular target, such as, for example, an “orphan” receptor about which the structure is known but the function or disease relevance is not known, is introduced to a screening process to be to be tested against the chemical compounds in the chemical compound database 300. Results of the screening process, including identification of chemicals that interacted with the new molecular target, are incorporated into the screening results database 200. Queries are made within database 100 to determine further steps to identify the function of the new molecular target and/or validate the disease relevance of the new target.

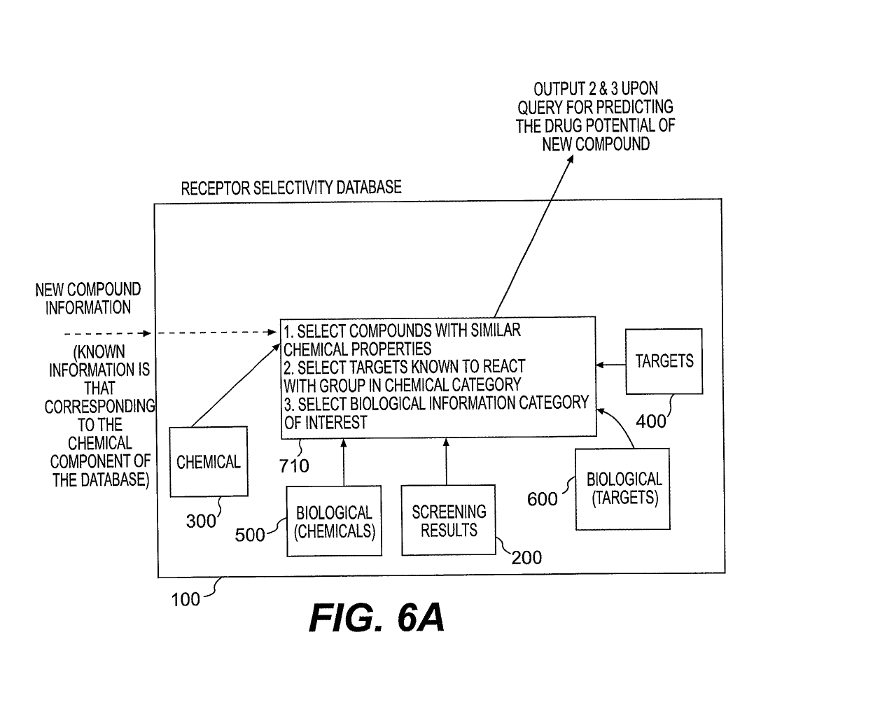

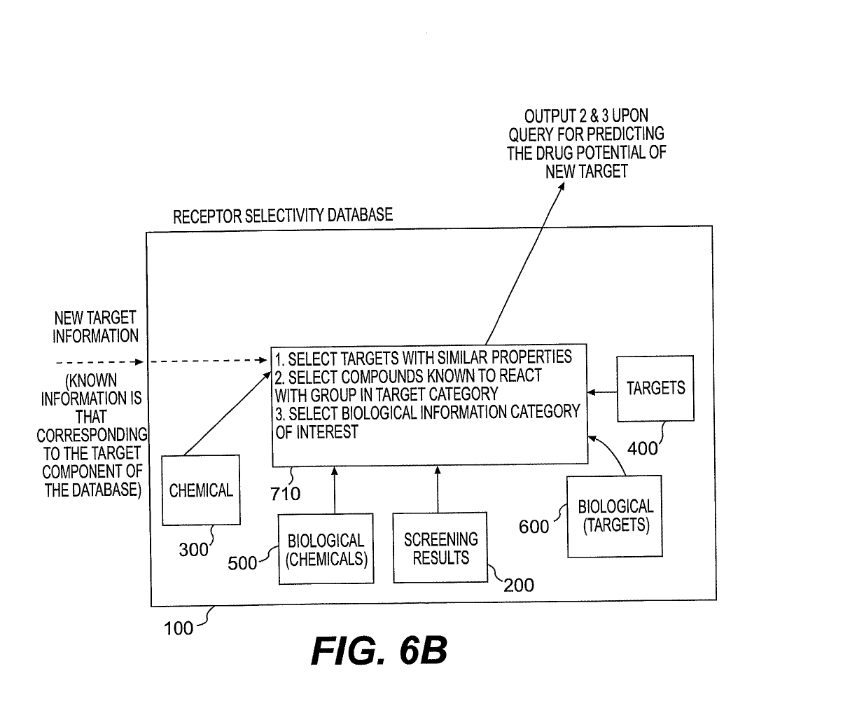

FIG. 6A illustrates the use of the database 100 for predicting the drug potential of a new compound. A logical table 710 relies on information from the chemical compound (300), molecular target (400), biological information (500 and 600), and screening results (200) databases. The table 710 is filled in with information from one or more of these databases (or tables) by executing an automatic query script to retrieve the information once a user provides the database 100 with information about a new chemical compound.

The query script used for the creation of table 710 may select chemical compounds from the chemical compound database 300 upon receiving the new compound information. The selection may be based on similar characteristics, such as chemical structure or other properties, between the new compound and the compounds already included in the database 300.

After the selection of chemical compounds, the query script selects targets from the target database 400 that are known to react (e.g., bind) with the selected compounds. Finally, the combination of selected chemical compounds and selected molecular targets may be used for querying the biological information databases 500 and 600 and inserting biological information corresponding to chemical compound-molecular target pairings into table 710. Alternatively, the user may enter a specific biological information category of interest (e.g., toxicity) so that the biological information included in table 710 is limited to that category.

The table 710 may be queried by the user to produce information relevant to the predictability of the potential use of the new compound as a drug. An example of this would be a query of the molecular targets known to react with chemical compounds associated with the new compound, and the known side effects produced by the chemical compounds when combined with the retrieved targets.

FIG. 6B illustrates the use of the database 100 to validate the disease relevance and/or the biological function of a new molecular target using an approach similar to that used to predict the drug potential of a new compound, but with the data inputs and queries shown in FIG. 6B.

The datasets described above have been used in the broad area of drug discovery and development pertaining, in particular, to the development of new treatment regimens for treating drug addiction and abuse. The utility of the invention in this area is a consequence of research on the biochemical processes involved in the human brain relating to such basic behaviors as pleasure, reward, excitement, fear, anxiety, sleep, etc. Central to these phenomena are the release from nerve cells, the extracellular activity, and the reuptake back into nerve cells of a group of neurotransmitter chemicals called catecholamines, which include dopamine, serotonin, and norepinephrine. The extracellular activity of these chemicals is primarily mediated by binding of the neurotransmitters to cell surface receptors, and the reuptake is accomplished by transporters that bridge through the cell membrane. Receptors for the neurotransmitters exist in numerous forms, or subtypes, and are distributed in different tissues and organs in the body.

Information concerning neurotransmitters and their receptors, in particular the biological activity of these compounds in relation to the human behaviors described above, has been incorporated in the present invention into a relational database together with chemical and biological activity information for a wide variety of chemical compounds. This database provides for methods to identify compounds that share certain chemical and physiological characteristics of abused or addictive compounds, which in turn provides a basis to determine new treatment regimens for patients who are abusing or are addicted to drugs.

Many different addictive drugs share a common physiological activity. Substances that make humans feel good all have a remarkably similar effect on a region of the brain called the “pleasure” or “reward” center. Nearly all of these substances have the capacity to increase the levels of dopamine in the nerve synapses in the “pleasure” center of the brain. Some substances have a direct effect on dopamine, others have an apparent indirect effect mediated by interactions between the substances and other types of receptors and transporters. The end result is the same, however. The feeling of pleasure resulting from the heightened levels of dopamine can lead to the behavior of “reward” by continuing to feed the brain with the pleasure-inducing substance to maintain the high dopamine levels. This is the essence of addiction.

There are numerous substances, or chemicals that are components of natural materials, that are subject to abuse and that on repeated use can become addictive. Dependency on such chemicals can have severe adverse psychological, societal, and economic impacts. The pleasure inducing substance can be cocaine, heroin, amphetamines (speed), nicotine, alcohol, barbiturates, marijuana, or any number of other drugs of abuse, or they can be pharmaceuticals intended to have other beneficial effects, or they can even be genetic, environmental, or behavioral factors themselves. So there are also numerous pharmaceuticals that, while performing a positive purpose as denoted by their therapeutic indication approved by regulatory authorities such as the Food and Drug Administration, can themselves become addictive on repeated dosing and may become abused.

While the end result is basically the same, the means is different. Blocking drug addiction for specific substances therefore requires an understanding of the complex mechanisms and interactions leading up to the elevated dopamine levels. Furthermore, since the perturbations associated with addiction are associated with effects common to a wide range of emotional or behavioral factors associated with numerous central nervous system diseases, understanding this complex set of targets can form the basis of finding improved drugs for treating diseases other than drug addiction, such as depression, attention deficit hyperactivity disorder, obesity or other eating or compulsive disorders, anxiety, etc., that also represent enormous potential markets and commercial opportunities.

One goal of pharmaceutical research and development is to discover and develop compounds or treatment regimens to combat drug addiction and dependency. One approach toward this goal has been to identify functional antagonists to the abused substance. An area in which this approach has been successfully employed is in the development and use of methadone to treat heroin addiction. Often, however, attempts to develop such treatment regimens have been hampered by a lack of understanding of the complex set of interactions between addictive substances and the molecular targets by which they exert their direct influence. The instant invention solves this problem by providing a relational database in which the interactions between addictive substances and molecular targets can be systematically studied and compared with information from a library of other chemical compounds to identify chemicals that share the same or similar biological activity profile as the addictive substance. Such chemicals, given their binding to molecular targets bound by the addictive substance, serve as candidates for new treatment regimens for patients suffering from addiction to the substance.

Cocaine addiction and dependency is one example of substance abuse that has been extensively studied. It has been widely reported (Smith 1999 and references therein) that cocaine addiction and dependency is attributable to its purported promiscuous interactions with the dopamine transporter and dopamine receptor subtypes, serotonin transporter and serotonin receptor subtypes, noradrenaline transporter and adrenergic receptors and subtypes, and other receptors and ion channels of a variety of subtypes. To date, there has been no reported success in identifying an effective chemical to block the action of cocaine. Nor has there been any demonstration of an agonist or an antagonist active at any individual molecular target that has proven to be an effective treatment for cocaine dependency and addiction.

The inventors have determined that cocaine is specifically, concurrently, and potently reactive with two transporters, namely the dopamine and serotonin transporters. We have further demonstrated that cocaine is also reactive, but only modestly, with the noradrenaline transporter, serotonin receptor subtype 3 (5HT3), and Sigma 1 receptors. Therefore, when selecting and designing treatment regimens for cocaine addiction and dependency, this invention provides that it is important to identify a set of the key relevant molecular targets. It is such a set of targets that can be effectively used as a guide for the selection and design of treatment regimens.

The present invention encompasses the determination of the biological activity profile of cocaine. The present invention also encompasses a method for determination and identification of the set of molecular targets that is critically useful in selecting and designing treatment regimens for addiction and dependency. The present invention further encompasses methods applicable for determining the selected set of the relevant targets for treating addiction and dependency based on a comprehensive database comprised of (1) biological activity profiles of chemical substances that cause abuse, addiction and dependency and (2) physiological and biochemical information on the targets against which these chemical substances have been tested.

The reported in vivo pharmacology associated with cocaine addiction and dependency, when annotated into the database with the chemical reactivity profile of cocaine and analyzed using the database, clearly demonstrates links to the physiology of all three transporters rather than that of the dopamine transporter alone. Cocaine produces a dosage dependent increase in heart rate and blood pressure that is accompanied by an increase in arousal, by improved performances on tasks, and by vigilance and alertness and by a sense of self-confidence and well-being. High dosages of cocaine produce euphoria, involuntary motor activity, stereotypic behavior, paranoia, and irritability and increased risk of violence.

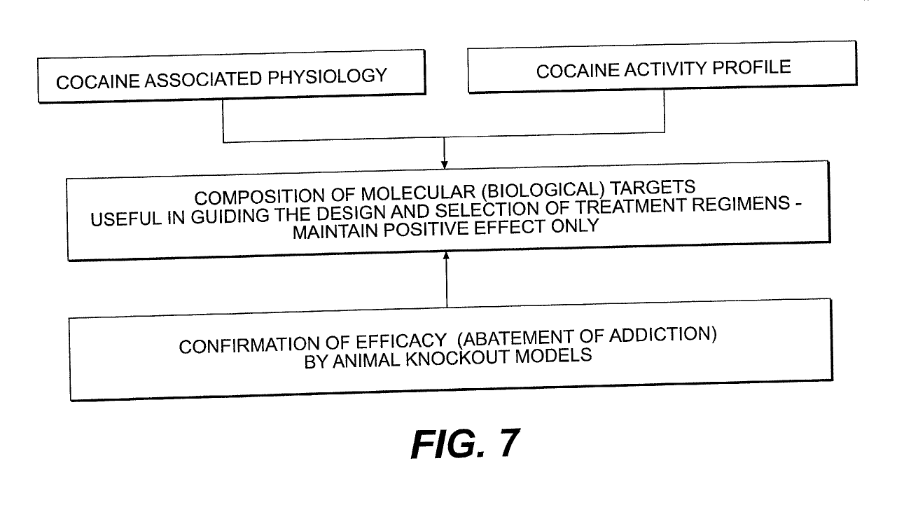

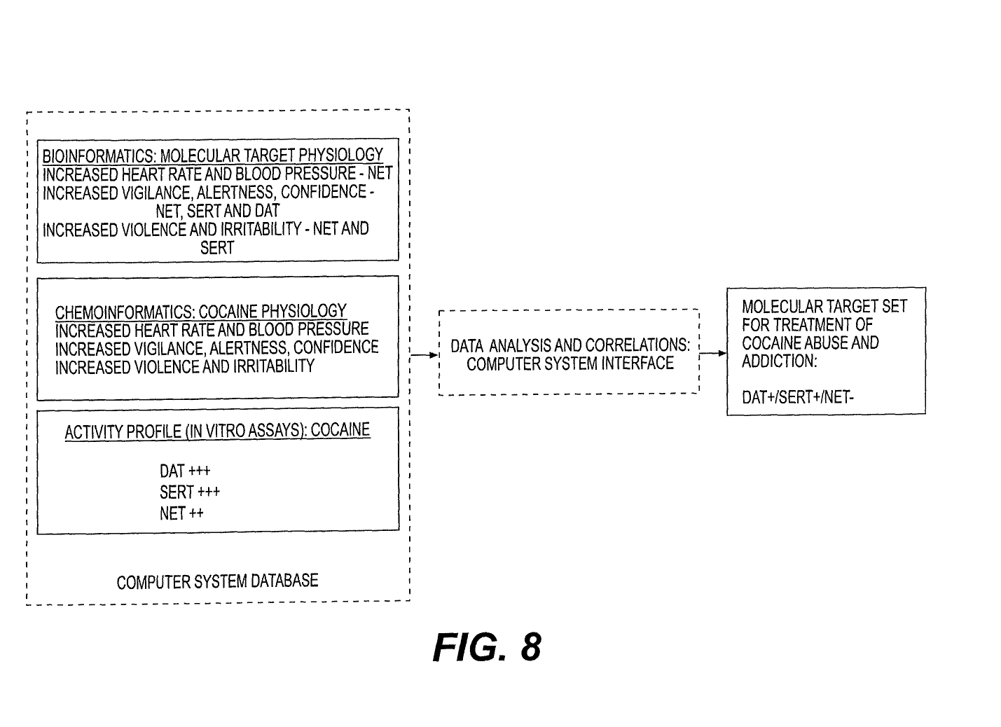

One strategy for analyzing the information in the database is presented in FIGS. 7 and 8.

Our strategy involves developing drug-based treatment regimens for cocaine (or other addictive and abused substances) that interact with a specific set of pharmacological targets. These regimens are directed to molecular targets at which the positive effects of the substance (for cocaine: the alertness, self-confidence and well being elements, i.e., pharmacologies associated with DAT and SERT) are evidenced, without the negative effects of the substance (for cocaine: violence, irritability, heightened sexual drive and cardiac effects, i.e., pharmacology that can be attributed to activity at NET).

This drug discovery approach with cocaine has been subsequently confirmed and reinforced in part by genetic knock-out animal models (Sora et al., Apr. 24, 2001). These models demonstrate that the elimination of both the DAT and SERT genes in mice, producing combined DAT and SERT knock-out mice, results in animals that do not develop an addiction to cocaine. Our earlier discovery identified a pharmacological equivalent of the phenomenology of this knock-out mouse model and has defined methods for using this information to identify compounds or combinations of compounds for use as a human pharmaceutical to treat cocaine addiction.

One embodiment of this invention is to establish an internally consistent and comprehensive biological activity-profile of cocaine (and other addictive chemical substances) by testing cocaine (and other addictive substances) against a wide and defined panel of biological targets, which forms the foundation of determining the causal pharmacology of cocaine (and other drug) dependency. The panel described in the examples consists of 131 different biological targets, which are listed in Table 1. Due to the abuse, addiction and dependency components of this disease, this panel should preferably be composed of primarily central nervous system-related receptors, ion channels, enzymes, and transporters.

TABLE 1

| Pass 1 | | | | |
|---|---|---|---|---|
| (10⁻⁵ M) | Assay Name/ | | | |
| % | Molecular Target | Ligand/ | Reference | Ref |
| Inhibition | (Source) | Substrate | Compound | Kⱼ |
| 32.33% | Orphanin (Human | [3H] | Nociceptin | 2.57E-9 |
| | Recombinant) | Nociceptin | | |
| 16.52% | Adenosine | [3H]-NBTI | NBTI | 5.40E-10 |
| | Transporter | | 2-Chloro- | |
| | (Human) | | adenosine | |
| 2.29% | Adenosine, A1 | [3H]CPX | (2-CADO) | 5.87E-8 |
| | | | 2-Chloro- | |
| | | | adenosine | |
| 26.30% | Adenosine, A2 | [3H]CGS21680 | (2-CADO) | 2.68E-8 |
| 25.92% | Adenosine, A2A | [3H]CGS21680 | NECA | 7.01E-8 |
| | (Human | | monohydrate | |
| | Recombinant)* | | | |
| −1.18% | Adrenergic, | [3H]-7- | Phentol- | 9.21E-9 |
| | Alpha 1A | MeOxy- | amine | |
| | | Prazosin | | |
| −6.30% | Adrenergic, | [3H]-7- | Phentol- | 3.04E-8 |
| | Alpha 1B | MeOxy- | amine | |
| | | Prazosin | | |
| 15.34% | Adrenergic, | [3H]MK-912 | Oxymeta- | 3.30E-9 |
| | Alpha 2A | | zoline HCl | |
| | (Human) | | | |
| −3.82% | Adrenergic, | [3H]MK-912 | Oxymeta- | 0.85E-8 |
| | Alpha 2B | | zoline HCl | |
| −5.94% | Adrenergic, | [3H]MK-912 | Oxymeta- | 9.29E-8 |
| | Alpha 2C | | zoline HCl | |
| | (Human | | | |
| | Recombinant) | | | |
| 11.74% | Adrenergic, | [125I]Iodo- | Alprenolol | 1.04E-9 |
| | Beta 1 (Human | cyanopindolol | | |
| | (Recombinant)* | | | |
| −15.59% | Adrenergic, | [125]I-Iodo- | Alprenolol | 5.41E-9 |
| | Beta 2 (Human | cyanopindolol | | |
| | (Recombinant)* | | | |
| 0.51% | Benzodiazepine, | [3H]PK 11195 | PK 11195 | 1.84E-9 |
| | peripheral | | | |
| | (Human) | | | |
| 33.44% | Cannabinoid, | [3H]-CP55940 | HU-210 | 3.00E-10 |
| | CB1 (Human | | | |
| | recombinant) | | | |
| 0.78% | Cannabinoid, | [3H]-CP55940 | HU-210 | 9.97E-10 |
| | CB2 (Human | | | |
| | recombinant) | | | |
| 1.37% | Clozapine | [3H]Clozapine | Clozapine | 3.98E-9 |
| 91.39% | Dopamine | [3H]WIN | GBR12909 | 1.32E-8 |
| | Transporter | 35428 | | |
| 17.51% | Dopamine, D1 | [3H]-SCH- | SCH23390 | 3.80E-10 |
| | (Human | 23390 | | |
| | Recombinant)* | | | |
| 30.57% | Dopamine, D2s | [3H]-Spiperone | Haloperidol | 2.08E-9 |
| | (Human | | | |
| | Recombinant)* | | | |
| 30.21% | Dopamine, D3 | [3H]7-OH- | (+/−)-7-OH- | 3.16E-10 |
| | (Rat | DPAT | DPAT HBr | |
| | Recombinant)* | | | |
| 18.75% | Dopamine, D4.4 | [3H]-YM- | Haloperidol | 1.96E-9 |
| | (Human | 09151-2 | | |
| | Recombinant)* | | | |
| 2.44% | Dopamine, D5 | [3H]-SCH- | R(+)-SCH- | 6.03E-10 |
| | (Human | 23390 | 23390 | |
| | Recombinant)* | | | |
| 5.81% | GABA A, | [3H]GABA | GABA | 9.46E-9 |
| | Agonist Site | | | |
| 9.58% | GABA A, BDZ, | [3H]Fluni- | Clonazepam | 7.00E-10 |
| | alpha 1, central | trazepam | | |
| 24.90% | GABA-B* | [3H]CGP- | (+/−) | 1.22E-6 |
| | | 54626A | Baclofen | |
| −9.30% | Glutamate, | [3H]AMPA | (+/−)AMPA | 1.52E-8 |
| | AMPA Site | | HBr | |
| 1.95% | Glutamate, | [3H]Kainic | Kainic Acid | 1.34E-8 |
| | Kainate Site | acid | | |
| 38.66% | Glutamate, | [3H]CGP | NMDA | 1.31E-5 |
| | NMDA Agonist | 39653 | | |
| | Site | | | |
| −1.56% | Glutamate, | [3H]-MDL- | MDL- | 1.63E-8 |
| | NMDA, Glycine | 105,519 | 105,519 | |
| | (Stry-insens | | | |
| | Site)* | | | |
| −9.99% | Glycine, | [3H]Strychnine | Strychnine | 1.36E-7 |
| | Strychnine- | | nitrate | |
| | sensitive | | | |
| −19.69% | Histamine, H1 | [3H]Pyrilamine | Triprolidine | 3.60E-9 |
| | | | HCl | |
| 28.75% | Histamine, H2* | [125I]-Amino- | Tiotidine | 8.70E-9 |
| | | potentidine | | |
| −4.60% | Histamine, H3 | [3H]N-a- | N-a-Methyl- | 1.31E-9 |
| | | MeHistamine | histamine | |
| | | | (NAMH) | |
| −0.78% | Imidazoline, I1 | [125I]- | Iodoclonid- | 7.89E-9 |
| | | Clonidine | ine | |
| 37.90% | Imidazoline, I2, | [3H]2-BFI | 2-BFI | 5.40E-11 |
| | central | | | |
| 16.13% | Melatonin | [125I]-2- | 2-Iodome- | 5.81E-11 |
| | | Iodomelatonin | latonin | |
| 23.28% | Muscarinic, M1 | [3H]Scopol- | (−)Scopol- | 6.00E-11 |
| | (Human | amine, | amine, MeBr | |
| | Recombinant)* | N-Methyl | | |
| 3.57% | Muscarinic, M2 | [3H]Scopol- | (−)Scopol- | 2.18E-10 |
| | (Human | amine, | amine, MeBr | |
| | Recombinant)* | N-Methyl | | |
| −8.07% | Muscarinic, M3 | [3H]Scopol- | (−)Scopol- | 1.88E-10 |
| | (Human | amine, | amine, MeBr | |
| | Recombinant)* | N-Methyl | | |
| 0.42% | Muscarinic, M4 | [3H]Scopol- | (−)Scopol- | 1.68E-10 |
| | (Human | amine, | amine, MeBr | |
| | Recombinant)* | N-Methyl | | |
| 6.01% | Muscarinic, M5 | [3H]Scopol- | (−)Scopol- | 4.49E-10 |
| | (Human | amine, | amine, MeBr | |
| | Recombinant)* | N-Methyl | | |
| 12.28% | Nicotinic (a- | [3H] | (+/−) | 5.91E-11 |
| | bungarotoxin | Epibatidine | epibatidine | |
| | insensitive) | | | |
| 60.04% | Norepinephrine | [3H]Nisoxetine | Desimipr- | 1.70E-9 |
| | Transporter | | amine HCl | |
| | | | (DMI) | |
| 2.51% | Opiate, Delta 1 | [3H]DPDPE | Naloxone | 4.80E-9 |
| | | | HCl | |
| 21.54% | Opiate, Delta 2 | [3H]-Naltrin- | Natriben | 3.38E-10 |
| | (Human | dole | methane- | |
| | Recombinant)* | | sulfonat | |
| 1.87% | Opiate, Kappa | [3H]- | Naloxone | 6.32E-9 |
| | (Human | Diprenorphine | HCl | |
| | Recombinant)* | | | |
| 10.19% | Opiate, Kappa 1 | [3H]U-69593 | U-69593 | 4.11E-10 |
| −4.22% | Opiate, Mu | [3H]DAMGO | Naloxone | 1.81E-9 |
| | | | HCl | |
| 3.08% | Opiate, Mu | [3H]- | Naloxone | 5.14E-10 |
| | (Human | Diprenorphine | HCl | |
| | Recombinant)* | | | |
| 16.43% | Purinergic, P2Y | [35S]-ATPas | ADPbS, | 2.12E-6 |
| | (Human)* | | Adenosine | |
| | | | b-thio-d | |
| 97.49% | Serotonin | [3H]- | Imipramine | 4.44E-9 |
| | Transporter | Citalopram | | |
| | (Human) | | | |
| 0.52% | Serotonin, | [3H]-8-OH- | (+/−)-8-OH- | 4.85E-9 |
| | 5HT1A | DPAT | DPAT HBr | |
| 7.63% | Serotonin, | [3H]-8-OH- | 8-OH-DPAT | 1.17E-9 |
| | 5HT1A (Human | DPAT | | |
| | Recombinant)* | | | |
| 3.31% | Serotonin, | [125I](−)- | Serotonin | 2.43E-8 |
| | 5HT1B | Cyanpindol, | | |
| | | iodo | | |
| 3.12% | Serotonin, | [3H]-5-CT | 5-carbox- | 2.59E-9 |
| | 5HT1D (Human) | | amidotrypt- | |
| | | | amine | |
| 23.35% | Serotonin, | [3H]- | Ketanserin | 1.27E-8 |
| | 5HT2A (Human) | Ketanserin | | |
| 1.44% | Serotonin, | [3H]- | Mianserin | 7.71E-10 |
| | 5HT2C | Mesulergine | HCl | |
| 60.28% | Serotonin, | [3H]GR 65630 | MDL 72222 | 8.30E-9 |
| | 5HT3 | | | |
| 22.12% | Serotonin, | [3H]GR | Serotonin | 3.65E-8 |
| | 5HT4 | 113808 | | |
| 11.98% | Serotonin, | [3H]-LSD | Methiothepin | 6.94E-9 |
| | 5HT5A (Human | | mesylate | |
| | Recombinant)* | | | |
| 1.13% | Serotonin, | [3H]-LSD | Methiothepin | 4.98E-10 |
| | 5HT6 (Human | | mesylate | |
| | Recombinant)* | | | |
| 26.77% | Serotonin, | [3H]-LSD | Methiothepin | 6.65E-10 |
| | 5HT7 (Human | | | |
| | Recombinant)* | | | |
| 64.70% | Sigma 1 | [3H]-(+)- | R(+)-3-PPP | 1.27E-9 |
| | | Pentazocine | HCl | |
| 38.83% | Sigma 2 | [3H]-DTG | Haloperidol | 1.25E-8 |
| 15.21% | Complement C5a | [125I]BH-rC5a | rC5a, | 6.19E-10 |
| | (Human) | | Human | |
| 12.80% | Estrogen | [125I]3,17B- | 17-B- | 1.06E-10 |
| | | Estradiol, 16a | Estradiol | |
| 20.02% | Glucocorticoid | [6,7-3H] | Triamcino- | 1.74E-9 |
| | | Triamcinolone | lone | |
| | | acetonide | | |
| −10.86% | Progesterone | [3H] | Prome- | 5.67E-9 |
| | | Promegestone | gestone | |
| −7.60% | Testosterone | [3H]Methyl- | Methyl- | 7.42E-10 |
| | (cytosolic) | trienolone | trienolone | |
| | | | (R1881) | |
| 20.28% | Calcium Channel, | [3H]Diltiazem, | Diltiazem | 7.63E-8 |
| | Type L | cis(+) | HCl | |
| | (Benzothiazepine | | | |
| | Site) | | | |
| 10.24% | Calcium Channel, | [3H] | Nifedipine | 6.22E-10 |
| | Type L | Nitrendipine | | |
| | (Dihydropyridine | | | |
| | Site) | | | |
| 11.51% | Calcium Channel, | [125I]- | w-Conotoxin | 1.24E-11 |
| | Type N | Conotoxin | GVIA | |
| | | GVIA | | |
| 8.40% | GABA, Chloride, | [3H]TBOB | TBPS | 1.55E-8 |
| | TBOB Site | | | |
| 5.36% | Glutamate, | [3H]Glutamic | L-Glutamic | 3.79E-7 |
| | Chloride | Acid | acid | |
| | Dependent Site | | | |
| −2.86% | Glutamate, | [3H]MK-801 | (+)-MK-801 | 2.33E-9 |
| | MK-801 Site | | HMaleate | |
| 6.63% | Glutamate, | [3H]TCP | (+)-MK801 | 8.96E-9 |
| | NMDA, | | Hydrogen | |
| | Phencyclidine | | Maleate | |
| | Site | | | |
| 7.31% | Potassium | [3H] | Glibencl- | 3.69E-10 |
| | Channel, ATP- | Glibenclamide | amide | |
| | Sensitive | | | |
| 11.04% | Potassium | [125I]Apamin | Apamin | 4.63E-11 |
| | Channel, Ca2+ | | | |
| | Act., VI | | | |
| 16.63% | Potassium | [125I] | Charybdo- | 2.04E-10 |
| | Channel, Ca2+ | Charybdotoxin | toxin | |
| | Act., VS | | | |
| 2.74% | Sodium, Site 1 | [3H]Saxitoxin | Tetrodotoxin | 3.36E-8 |
| 36.80% | Sodium, Site 2 | [3H]Batracho- | Aconitine | 1.30E-6 |
| | | toxin A 20-a | | |
| | | Benzo | | |
| 6.27% | Ahenylate | [3H]Forskolin | Forskolin | 3.38E-8 |
| | Cyclase, | | | |
| | Forskolin | | | |
| −19.14% | Inositol | [3H]IP3 | IP3 | 1.53E-8 |
| | Triphosphate, IP3 | | | |
| −15.74% | NOS (Neuronal- | [3H]NOARG | NOARG | 3.22E-8 |
| | Binding) | | (Nitro-L- | |
| | | | Arginine) | |
| −4.44% | Protein Kinase C, | [3H]PDBu | PDBu | 7.64E-9 |
| | PDBu | | | |
| 2.79% | Adenosine | [3H]- | NBTI | 3.31E-10 |
| | Transport | Adenosine | | |
| | (cs + es) | | | |
| | (Human) | | | |
| 47.47% | Adenosine | [3H]- | NBTI | 1.16E-8 |
| | Transport | Adenosine | | |
| | (es) (Human) | | | |
| −19.12% | Choline Transport | [3H]Choline | Choline | 1.59E-5 |
| | | chloride | chloride | |
| −19.43% | GABA Transport | [3H]GABA | (+/−) | 1.33E-5 |
| | | | Nipecotic | |
| | | | acid | |
| 24.20% | Glutamate | [3H]Glutamic | D-Aspartic | 6.54E-6 |
| | Transport | Acid | Acid | |
| 17.06% | Leukotrine B4, | [3H]LTB4 | LTB4 | 5.26E-10 |
| | LTB4 | | | |
| −17.11% | Leukotrine D4, | [3H]LTD4 | LTD4 | 8.85E-9 |
| | LTB4 | | | |
| 22.14% | Thromboxane A2 | [3H]SQ 29,548 | Pinane- | 3.03E-8 |
| | (Human) | | thromboxane | |
| | | | A2 | |
| 11.36% | Atrial Natriuretic | [125I]ANP | tANP (rat) | 1.22E-10 |
| | Peptide, ANP A | (Rat) | | |
| 22.49% | Corticotropin | [125I]Tyr0- | Tyr0-oCRF | 7.38E-9 |
| | Releasing Factor, | oCRF | | |
| | CRF | | | |
| 10.05% | Epidermal | [125I]EGF | EGF | 3.20E-9 |
| | Growth Factor, | | | |
| | EGF | | | |
| −4.65% | Oxytocin | [3H]Oxytocin | Oxytocin | 8.61E-10 |
| 12.58% | Platelet | [3H]Hexa- | C16 PAF | 7.69E-9 |
| | Activating Factor, | decyl, PAF | | |
| | PAF | | | |
| −11.95% | Thyrotropin | [3H]- | (3MeHis2) | 1.75E-7 |
| | Releasing | (3MeHis2)TRH | TRH | |
| | Hormone, TRH | | | |
| 21.23% | Angiotensin II, | [125I]-(Sar1- | Angiotensin | 2.14E-8 |
| | ATI (Human) | Ile8) | II (Human) | |
| | | Angiotensin | | |
| 5.85% | Angiotensin II, | [125I]-Tyr4 | Angiotensin | 5.78E-10 |
| | AT2 | Angiotensin II | II (Human) | |
| 4.48% | Bradykinin, BK2 | [3H]- | Bradykinin | 7.00E-10 |
| | (Human | Bradykinin | TFA | |
| | recombinant) | | | |
| 11.15% | Cholecystokinin, | [125I]CCK-8 | CCK-8 | 2.28E-11 |
| | CCK1 (CCKA) | | (sulfated) | |
| −14.06% | Cholecystokinin, | [125I]CCK-8 | CCK-8 | 4.79E-10 |
| | CCK2 (CCKB) | | (sulfated) | |
| 0.19% | Endothelin, ET-B | [125I] | Endothelin-1 | 1.91E-10 |
| | (Human | Endothelin | | |
| | Recombinant)* | (porcine) | | |
| | NA | | | |
| −14.73% | Galanin | [I125]Galanin | Galanin | 1.94E-10 |
| | | | (Porcine) | |
| −1.72% | Neurokinin, NK1 | [3H]Substance | Substance P | 1.30E-8 |
| | | P | | |
| 13.71% | Neurokinin, NK2 | [125I]-NKA | Neurokinin | 7.73E-10 |
| | (NKA) (Human | | A | |
| | Recombinant)* | | | |
| −15.24% | Neurokinin, NK3 | [125I]Eledoisin | Eledoisin | 5.48E-9 |
| | (NKB) | | | |
| 26.58% | Neuropeptide, | [125I]PYY | NPY | 3.45E-9 |
| | NPY1 (Human) | | (porcine) | |
| 4.27% | Neuropeptide, | [125I]-PYY | NPY | 3.75E-9 |
| | NPY2 (Human) | | (Human, rat) | |
| 19.14% | Neurotensin | [125I]Neuro- | Acetyl-NT | 3.40E-10 |
| | (Human | tensin | (8-13) | |
| | Recombinant) | | | |
| −3.54% | Somatostatin, | [125]- | Somatostatin | 7.68E-10 |
| | Non-selective | Somatostatin- | | |
| | | 14 (Tyr11) | | |
| 30.71% | Vasoactive | [125I]VIP | VIP | 1.90E-9 |
| | Intestine Peptide, | | | |
| | Non-selective | | | |
| 0.98% | Vasopressin 1 | [3H]Vaso- | Arg8- | 9.98E-10 |
| | | pressin-1 | Vasopressin | |
| | | Antagonist | (AVP) | |
| −4.37% | Vasopressin, | [125I]-Via | (Phe)(Me) | 1.28E-10 |
| | V1A (Human)* | Antagonist | 2A6,8, | |
| | | | L9AVP | |
| 8.18% | Acetyl- | Acetyl- | Eserine | 9.25E-7 |
| | cholinesterase | thiocholine | (Phy- | |
| | | | sostigmine) | |
| 8.25% | Choline | [14C]Acetyl | beta-NETA | 4.25E-7 |
| | Acetyltransferase | Coenzyme | | |
| −11.24% | Elastase (Human) | MeO-Suc-Ala- | Ursolic Acid | 2.32E-6 |
| | | Ala-Pro-Val- | | |
| | | pNA | | |
| 31.58% | Esterase (Human) | Acetyl- | Eserine | 1.22E-6 |
| | | thiocholine | | |
| 25.43% | GABA | [14C]-GABA | Amino- | 0.00E-1 |
| | Transaminase | | oxyacetic | |
| | | | acid | |
| −1.39% | Glutamic Acid | [14C]Glutamic | AminoOxy | 5.04E-10 |
| | Decarboxylase | acid | acetic acid | |
| 32.79% | Monoamine | [14C]-5HT | Ro 41-1049 | 1.08E-9 |
| | Oxidase | (Serotonin) | HCl | |
| | A, Peripheral | | | |
| −22.82% | Monoamine | [14C]Phenyl- | Ro 16-6491 | 1.20E-8 |
| | Oxidase | ethylamine | HCl | |
| | B, Peripheral | | | |
| −11.51% | NOS | [3H]Arginine | L-Arginine | 3.00E-5 |
| | (Constitutive- | | | |
| | Neuronal) | | | |
| 15.33% | Protein | DiFMUP | Calyculin | 7.22E-10 |
| | Phosphatase, | | | |
| | PP2A (Human) | | | |
| −10.37% | Protein | pNPP | ammonium | 9.05E-5 |
| | Phosphatase, | | molybdate | |
| | PP2B | | | |
| | (Calcineurin) | | | |
| 65.03% | Protein Tyrosine | pNPP | Ammonium | 3.13E-7 |
| | Phosphatase, | | Molybdate | |
| | PTP-B (Human) | | | |
| 12.01% | Protein Tyrosine | pNPP | Ammonium | 3.09E-8 |
| | Phosphatase, | | Molybdate | |
| | PTP-CD45 | | | |
| | (Human) | | | |
| 24.35% | Protein Tyrosine | pNPP | Ammonium | 1.17E-7 |
| | Phosphatase, | | Molybdate | |
| | PTP-LAR-D1 | | | |
| | (Human) | | | |
| −17.63% | Protein Tyrosine | pNPP | Ammonium | 4.13E-8 |
| | Phosphatase, | | Molybdate | |
| | PTP-Cell | | | |
| | (Human) | | | |

*Pass 1 activity data not confirmed for PTP-B in QA/QC follow-up

Pass 1 (10⁻⁵ M) percent inhibition data are shown for each assay (column 1). Also shown for each assay are the general source of the molecular target for each assay (“human” from human cell lines; “human recombinant” from human gene expressed in cloned cell lines; “rat recombinant” from rat gene expressed in cloned cell lines; no other source notation after assay name denotes animal cell or animal tissue source) (column 2); labeled ligand or substrate for each assay (column 3); and reference compound used for each assay (column 5), as well as (column 5) the experimental Ki for the reference compound used for each assay as a measure of QA/QC.

One may also define the pharmacological activity profile of cocaine based on those activities meeting a selected modest potency threshold of greater than 50% inhibition at 10⁻⁵ M (shown in Table 2) or as those activities meeting a selected highest threshold of greater than 75% inhibition at 10⁻⁵ M. These results are shown in Table 2.

TABLE 2

| | % Inhibiton | |
|---|---|---|
| Molecular Targets (Assays) | (10⁻⁵ M) | Ki Determination |
| Highest potency (>75% inhibition) | | |
| Serotonin Transporter (human) | 97.49 | 3.25 × 10⁻⁷ M |
| Serotonin Transporter (rat) | 96.16 | 3.52 × 10⁻⁷ M |
| Dopamine Transporter | 91.39 | 3.21 × 10⁻⁷ M |
| Modest potency (>50% but <75%) | | |
| Sigma 1 Receptor | 64.70 | 1.16 × 10⁻⁵ M |
| Serotonin 5HT3 Receptor | 60.28 | 3.09 × 10⁻⁶ M |
| Norepinephrine Transporter | 60.04 | 1.00 × 10⁻⁶ M |

The highest potency for cocaine was observed at the serotonin and dopamine transporters. Modest potency was also seen at the sigma 1 and serotonin 5HT3 receptors and norepinephrine transporter. The percent inhibition shown in the second column is for Pass 1 (10⁻⁵ M). The third column shows the Ki as determined from an analysis of the Pass 3 screening. Note that the relative potencies determined in Pass 1 were confirmed by the Ki determinations in Pass 3 for the two sets of targets.

Thus, contrary to conventional belief of those in the art, cocaine reacts specifically with only a few molecular targets that are mostly transporters. A few characteristics stand out from the profile. Cocaine demonstrates the highest potency activity against both dopamine and serotonin transporters, with little selectivity and preference between these two. Simultaneously, a reduced but noticeable inhibition (at 10⁻⁵M) against norepinephrine or noradrenaline transporter is also present. Other in vitro pharmacological characteristics of cocaine that are more subtle include the involvement of two other classes of specific receptors, namely the sigma and serotonergic receptors. The biological significance of sigma receptors has recently been recognized and reported with respect to glucose utilization, and in neurodegeneration, as well as in psychosis, depression, anxiety episodes and related diseases (Nabeshima, 1999). More importantly, chemicals such as Phencyclidine and Dizocilpine may have effects on these classes of receptors in that they modulate dopamine release (Ault, D. T and Werling L. L., 2000; 1999; Gudelsky, G A. 1999; Okuyama, S. 1999; Weatherspoon and Werling, 1999). Consequently the effects of cocaine that appear to be associated with dopamine's biological effect may in fact be mediated in part through the sigma receptors.

Data such as these may be used in conjunction with chemoinformatic information. The biological profile of cocaine may be compiled into a relational database along with the related and extensive chemoinformatic information on cocaine and other addictive compounds. The chemoinformatic information may include that obtained from the field of chemistry and related fields such as chemical structural information, physical chemistry, chemical purity, solubility, logP, chirality, etc. It may also include the in vivo biochemical and physiological effects and responses of the given chemical (for example, cocaine) that are known and reported in the scientific literature. For instance, related to cocaine, the field of annotation for “physiological response” might consist “increase in heart rate and blood pressure, accompanied by increase in arousal, improved performances on tasks, and vigilance and alertness and a sense of self-confidence and well being. High dosages of cocaine produce euphoria, involuntary motor activity, stereotypic behavior, paranoia, irritability and increased risk of violence”. A more detailed annotation may also include additional information, for instance “short and immediate exposure may induce prolonged and intense orgasm; long term exposure reduces sexual drive”.

Another embodiment of the invention is the bioinformatic annotations in the database.

The database contains extensive bioinformatic information covering all molecular targets used for profiling the chemical substances. The bioinformatic information may include that known to the art of biology and biochemistry and structural biology, such as peptide sequence, name, and structural class (7-transmembrane protein, globular protein, etc). It may also include information that is related to the physiological phenomena and pharmacological functions associated with the biological target. For instance, the annotations related to the noradrenaline transporter include information such as inhibition of reuptake causes increase in heart rate and blood pressure, (i.e., cardiac arrhythmias and increased systolic arterial pressure). As another example, the physiological information related to serotonin reuptake inhibition (blockage of serotonin transporter activity) may lead to “arousal/sedation, general well-being”. The information for the serotonin transporter also includes annotations such as “targets for treatment of schizophrenia, paranoia, and depression”.

Another aspect of the invention, as pertains to the database, is the nature of the relationship between the datasets comprising the database. That is, the relationships between chemicals and biological targets are not only linked through their interactions in terms of in vitro activity or potency, but also by their relationships in terms of physiological responses.

Still another embodiment is the selection of a preferred set of the molecular targets that are essential in treating cocaine addiction. The determination of the preferred set of targets that are useful in designing therapeutic regimens or in guiding selection of novel therapeutic compounds or combination of compounds is carried out using a relevant dataset derived from the above database. The biochemical and physiological information related to each of the included biological targets is annotated in the database. When these annotations are analyzed in light of its biological activity profile, one sees that cocaine addiction and dependency are the result of an individual chemical (cocaine) simultaneously and specifically interacting with three transporter systems (dopamine, serotonin and noradrenaline or norepinephrine) with different interacting potencies. The design and discovery of treatment regimens for cocaine addiction, abuse and dependency hence must take into consideration all three transporters, especially when the prior efforts by others related to any single target has not rendered any beneficial effect. Furthermore, according to the database annotations the sigma-1 receptor appears to be associated with the dopaminergic system, whereas the serotonin 5HT3 receptor is part of the serotonergic system. The sigma-1 receptor and serotonin 5HT3 receptor activities of cocaine again point to the dopamine and serotonin transporter systems as key players in defining the pharmacology underlying cocaine addiction.

Still another embodiment of this invention is to select or design a treatment regimen using a guide comprised of compounds having a positive interaction with the dopamine and serotonin transporters and absence of, or substantially reduced, molecular interaction with the noradrenaline transporter. According to the database and a conventional understanding of the receptor-related physiology, the blockage of the noradrenaline transporter will lead to a condition known as “adrenaline rush” which dramatically and often dangerously affects cardiovascular systems in terms of causing cardiac arrhythmias as well as a significant increase in systolic arterial pressure. Both symptoms are often observed and associated with cocaine overdose. Therefore, a primary consideration in designing a replacement therapy to treat cocaine addiction must address the issue of such a medication inhibiting the dopamine and serotonin transporters while having little or no effect on the noradrenaline transporter.

### EXAMPLE 1

### Preparation of Cocaine Solutions and Repositories for Profiling the Activity of Cocaine in Multiple Assays

Cocaine was stored at room temperature until use. At the initiation of Profiling, cocaine was dissolved in a small quantity of neat dimethyl sulfoxide (DMSO) and diluted to a working stock (10⁻⁴ M) of 4% DMSO (v/v) using distilled H₂O. For first pass screening (determination of activity at one concentration at the high end of physiological relevance; Pass 1) in individual assays, this compound solution was prepared as a compound repository for addition into the assay solution at a 1:10 ratio such that a final concentration of cocaine of 10⁻⁵ M was achieved.

Subsequently, for second pass screening for those assays in which cocaine demonstrated activity above a specified threshold (see Example 8 below) in Pass 1 screening, the working stock of 10⁻⁴ M cocaine in 4% DMSO was serially diluted using 4% DMSO to obtain solutions with working concentrations of 10⁻⁶ and 10⁻⁸ M. These solutions were added at 1:10 ratios to individual assays in order to achieve the desired final concentrations (10⁻⁹, 10⁻⁷ and 10⁻⁵ M) for second pass (Pass 2) screening, which was designed to establish preliminary evidence of concentration-dependent activity of cocaine in the assays.

For third pass screening for those assays in which cocaine demonstrated activity above a specified threshold (see Example 8 below) in Pass 1 or Pass 2 screening at 10⁻⁵ M, the working stock of 10⁻⁴ M cocaine in 4% DMSO was serially diluted using 4% DMSO to obtain solutions with a range of nine working concentrations. The specific concentrations in this range for each assay were selected based on the Pass 2 screening results of preliminary concentration dependence. These nine different solutions were added at 1:10 ratios to individual assays in order to achieve the desired final concentrations.

For all screening (Passes 1-3) repositories of cocaine working solutions were made at the beginning of each week, stored at room temperature and used throughout the week. New repositories were made on a weekly basis. One ordinarily skilled in the art will recognize that solvents other than DMSO, specific concentrations and numbers of concentrations in each range, number of screening passes, and other aspects of building repositories of cocaine solutions for screening purposes can be used without changing the nature of the invention.

### EXAMPLE 2

### Profiling Cocaine Activity at Multiple Targets

Working solutions of cocaine in DMSO were transferred to assay tubes or wells for each of a multitude of different assays. In this example, the assays were primarily designed to measure activity of compounds, such as cocaine, at receptors, transporters, ion channels, or enzymes that are molecular targets relevant to drug discovery and development, to chemical addiction or dependency, or to other aspects of biological systems and biological or chemical activity. In this example, a total of 131 different assays, each based on a different specific receptor, transporter, ion channel, or enzyme, were performed in order to obtain a profile of activity of cocaine. In general, each of these assays comprises a buffer solution, a cell or tissue preparation containing the specific molecular target, a labeled compound that is known to interact with and have biological activity at, the specific molecular target, and other assay components.

The buffer solution is selected to provide those conditions conducive to the desired reactivity measurements embodied in the specific assay.

The preparation of the receptor, transporter, or enzyme for the assay can be from animal or human tissues, from cells cultured in a laboratory and natively expressing the desired molecular target, or from cells or tissues transformed or transfected with a gene codifying the molecular target such that a recombinant or cloned form of the molecular target is produced. Such preparations can be crude, partially purified, or highly purified, depending on the characteristics of the assay. They could also consist of whole intact cells, tissues, or organs.

The labeled compound known to be active at the molecular target can be a small organic molecule, a peptide, a nucleic acid, an oligosaccharide, or a macromolecule such as a protein, polysaccharide, DNA, RNA, etc. The compound can be labeled with radioactivity such as ³H, ¹⁴C, ¹²⁵I, ³²P, or other isotopes; it can have a fluorescent, bioluminescent, or chemoluminescent tag; or it can have some other detectable or measurable characteristic, such as UV or visible absorbance, to allow the potential activity of the test compound (e.g., cocaine) to be determined. In such assays, the labeled compound is selected to have an interaction with the specific molecular target, such as a ligand that binds to a receptor, a substrate for an enzymatic reaction, a chemical that binds to an ion channel or transporter such that it alters its function, etc. Or in some assays the molecular target can be labeled, both the labeled compound and molecular target can be labeled, or neither can be labeled but the assay design is such that the interaction between the test compound and molecular target can be detected in a reproducible and preferably quantitative manner with and/or without any labeling and tagging.

In addition to assays designed to measure binding, inhibition, or other forms of molecular interaction, functional assays can be used to detect activity of compounds such as cocaine at specific targets. These functional assay formats could include whole cells that contain the specified target and that respond either chemically or biologically when exposed to a test compound, such as cocaine, that has biological activity at the target. These assays include such formats as cells transfected with the gene for a specific molecular target in such a way that active compounds induce a detectable signal such as a chemo- or bio-luminescent output (e.g., reporter gene assay) or a morphological or calorimetric change (e.g., melanophore assay). Or the functional assays can be based on detection of a secondary signal (such as cAMP, Ca++ flux, membrane depolarization, IP3 turnover, neurotransmitter release or ion transport, etc.).

All of these general concepts of assay design and components of assays are well known to those skilled in the art, and one would recognize that alternative formats of assays could be performed within the scope of this invention to achieve the end result of measuring the profile of activity of cocaine or other addictive compounds or other chemicals of interest.

All assays are performed with a concurrent set of controls, including testing one or more reference compounds with known activity at the specific molecular target. Reference compounds are tested for activity at multiple concentrations such that a Kᵢ or Kₘ can be calculated for each particular assay run. In order for high quality data to be entered into the database of activity of compounds such as cocaine at specific targets, the experimental constant for the reference compound should be within the accepted historical range for that assay. Preferably the maximum deviation for acceptable data quality is a Kᵢ or Kₘ that is within 0.5 log units of the historical values for the reference compound. Other quality control measures are performed on each assay run including positive and/or negative controls and blanks on each run, preferably on each assay plate.

In this example, most of the 131 assays used to profile the activity of cocaine are designed to measure receptor-binding, enzyme inhibition, or chemical inhibition of neurotransmitter transporters and ion channels. Due to the abuse, addiction and dependency components of cocaine use in humans, this assay panel is preferably composed of primarily central nervous system related receptors, ion channels, enzymes and transporters. Specific assay protocols for five of the 131 assays are summarized in Examples 3-7 below. These five were selected as those for which a selected threshold of 50% inhibition at 10⁻⁵ M in Pass 1 or Pass 2 was exceeded for cocaine activity. A full list of the 131 assays specifying the molecular targets and other key assay parameters of assay design, as well as Pass 1 data on the activity of cocaine at these targets, is shown in Table 1.

Ki values determined from Pass 3 data, where applicable, are shown in Table 2. The assay protocols for each of these 131 assays in Table 1 are published in the NovaScreen Short Assay Protocol book that is routinely provided to clients of NovaScreen's service business. This Short Assay Protocol Book is incorporated herein by reference in its entirety. Reproduction of the protocols in these examples was omitted for brevity but one ordinarily skilled in the art could take the information provided in NovaScreen's Short Assay Protocols and perform these assays in order to measure the activity of cocaine. Although the protocols reproduced in these Examples are limited to those at which cocaine demonstrated activity in excess of the 50% inhibition threshold, the data for cocaine activity at each of the 131 targets are very important to the present invention and use of the screening database since negative (or below threshold) activity at specified targets is also an important parameter for selecting new therapeutic compounds using the database for potential to treat cocaine addiction.

### EXAMPLE 3

### Assay for Dopamine Transporter Activity.

Cocaine (and other addictive chemicals or other compounds added to the database) were prepared as described in Example 1 and tested for activity at the dopamine transporter according to the following protocol. One skilled in the art would recognize that other assay protocols or modifications to the protocol below could provide the same type of information regarding determination of the activity of addictive or other chemicals at the dopamine transporter molecular target.

The assay is derived from Protocol for Dopamine Transporter Binding Assay (Javitch, J. J., Blaustein, R. O., and Snyder, S. H. [³H]Mazindol Binding Associated with Neuronal Dopamine and Norepinephrine Uptake Sites. Mol Pharmacol. 26: 35-44 (1984).)

Tissue Preparation

1. Frozen brains from male Guinea Pigs were thawed and placed in 50 mM TRIS-HCl (pH 7.4 at 25° C. with 120 mM NaCl). The striatum was isolated.

2. A Polytron was used to homogenize tissue in 20 vols. (w/v) of 50 mM Tris-HCl (pH 7.4 at 25° C. with 120 mM NaCl).

3. The homogenate was centrifuged at 48,400× g for approximately 10 minutes at 4° C. The supernatant was discarded.

4. The pellet was washed an additional time as described in steps 2 and 3.

5. The pellet was stored on ice until needed for binding assay.

6. Using a Polytron (setting 5; approximately 10 seconds) the pellet was resuspended in 50 mM Tris-HCl (pH 7.4 at 25° C. with 120 mM NaCl) to an initial concentration of 10 mg/ml (original wet weight), such that the final concentration was 8 mg/ml or 4.0 mg tissue/tube.

Material and Reagents

1. [³H]-WIN 35-428 was diluted to a concentration of 20 nM in 50 mM TRIS HCl (pH 7.4 at 25° C. with 120 mM NaCl). Thus, the final ligand concentration was 2.0 nM.

2. Non-specific binding was defined as that remaining in the presence of 1×10⁻⁶ M GBR13109 (room temperature). GBR13109 has a MW=523.5 g/mol and will solubilize in dH20.

3. The reference compound for the assay was GBR13109 and was diluted in 4% DMSO and then run at the following final concentrations: 1×10⁻¹⁰, 2×10⁻¹⁰, 5×10⁻¹⁰, 1×10⁻⁹, 2×10⁻⁹, 5×10⁻⁹, 1×10⁻⁸, 2×10⁻⁸, 5×10⁻⁸, 1×10⁻⁷, 2×10⁻⁷, 5×10⁻⁷ M.

4. The positive control GBR13109 was run at the final concentrations of: 2×10⁻⁸, 1×10⁻⁷, 5×10⁻⁷M.

5. The K_{d} for the receptor was 28.0 nM.

Binding Reaction

1. Each tube or well or any container of similar function received the following components:

50 ul drug or vehicle

50 ul [³H]-WIN 35,428

400 ul tissue suspension.

2. The binding reaction was initiated with the addition of tissue, and incubated for 120 minutes at 0° C. (on ice).

3. The binding reaction was terminated by rapid filtration of tube/well contents onto untreated Whatman GF/C filters. (filters dipped in wash buffer just prior to filtration).

4. The assay tubes were rinsed once with ice cold 50 mM TRIS HCl (pH 7.4 at 25° C. with 120 mM NaCl, 0.1% BSA), then the filters were rapidly rinsed with 6×1 mls/tube of the same wash buffer.

5. Radioactivity trapped onto the filters was assessed using a TopCount scintillation counter. Filters were dried overnight or placed in an oven, then wells were filled with scintillation fluid. The plate was allowed to sit for 1 hour before counting.

### EXAMPLE 4

### Assay for Serotonin Transporter Activity

Cocaine (and other addictive chemicals or other compounds added to the database) were prepared as described in Example 1 and tested for activity at the serotonin transporter according to the following protocols. One ordinarily skilled in the art would recognize that other assay protocols or modifications to the protocol below could provide the same type of information required regarding determination of the activity of addictive or other chemicals at the serotonin transporter molecular target. As further demonstration of differences in assay protocols yielding similar information with respect to determination of activity, two different protocols are described in this Example 4. The first uses rat brain as a source of the serotonin transporter and the second uses human cell-(platelet-) derived serotonin transporter. Pass 1 and Ki data for both the rat serotonin transporter and human serotonin transporter are shown in Table 2 and are nearly identical. Table 1 contains Pass 1 data for only the human serotonin transporter.

The assay is derived from D'Amato, R. J., Largent, B. L., Snowman, A. M., and Snyder, S. H. Selective Labeling of Serotonin Uptake Sites in Rat Brain by [³H] Citalopram Contrasted to Labeling of Multiple Sites by [³H] Imipramine. Jrn. Pharmacol. & Exp. Ther. 242: 364-371 (1987) with modifications.

Tissue Preparations

1. The brains from male Sprague-Dawley rats were removed immediately following decapitation and placed in ice cold 50 mM TRIS-HCl, containing 120 mM NaCl and 5 mM KCl (pH 7.4 at 25° C.). The forebrain region was isolated by dissection.

2. Using a Polytron (setting 5; approximately 10 seconds), the tissue was homogenized in 30 volumes of ice cold 50 mM TRIS-HCl containing 120 mM NaCl and 5 mM KCl (pH 7.4 at 25° C.).

3. The tissue homogenate was centrifuged at 48,400× g at 4° C. for 10 minutes. Supernatant was discarded.

4. The pellet was washed two more times as described in steps 2 & 3 for a total of 3 spins.

5. The pellet was stored on ice until needed for the binding assay.

6. Using a polytron (setting 5; approximately 10 seconds), the pellet was resuspended in 50 mM TRIS-HCl containing 120 mM NaCl and 5 mM KCl (pH 7.4 at 25° C.) to an initial concentration of 13.0 mg (original wet weight)/ml, such that the final tissue concentration was 10.4 mg/ml or 2.6 mg/tube.

Binding Reaction

1. Each tube/well received the following components:

25 ul drug or vehicle

25 ul [³H]-Citalopram

200 ul tissue suspension

2. The binding reaction was initiated with the addition of tissue, and incubated for 60 minutes at 25° C.

3. The binding reaction was terminated by rapid vacuum filtration of tube contents onto presoaked (0.5% PEI for at least 1 hour) GF/C filters.

4. The tubes were rinsed once with ice cold 50 mM TRIS-HCl containing 120 mM NaCl and 5 mM KCl (pH 7.4 at 25° C.), then the filters were rapidly rinsed with approximately 7 ml/tube of the same ice cold wash buffer.

5. Radioactivity trapped on the filters was assessed using a Beta Plate Scintillation Counter. Filters were allowed to soak in BetaScint for one hour prior to counting.

Material and Reagents

1. [³H]-Citalopram was diluted in 50 mM TRIS-HCl containing 120 mM NaCl and 5 mM KCl (pH 7.4 at 25° C.) to a concentration of 7 nM, such that the final radioligand concentration in the assay was 0.7 nM.

2. Non-specific binding was defined as that remaining in the presence of imipramine at 1×10-5M. Imipramine was solublized in dH₂O.

3. The reference compound was Imipramine run at final concentrations of: (Imipramine was solublized in dH₂O) 5×10⁻¹⁰, 1×10⁻⁹, 2×10⁻⁹, 5×10⁻⁹, 1×10⁻⁸, 2×10⁻⁸, 5×10⁻⁸, 1×10⁻⁷, 2×10⁻⁷, 5×10⁻⁷, 1×10⁻⁶, 2×10⁻⁶ M.

4. The positive control was Imipramine run at final concentrations of 2×10⁻⁸, 1×10⁻⁷, and 1×10⁻⁶ M.

5. The K_{d} of the serotonin transporter receptor for [³H]-Citalopram was 1.7 nM.

| Buffers | | MW (g/mole) | for 4 Liters |
|---|---|---|---|
| Assay Buffer: | 50 mM TRIS-HCl | 121.14 | 24.23 g |
| | (pH 7.4) | | |
| | 120 mM NaCl | 58.44 | 28.05 g |
| | 5 mM KCl | 74.55 | 1.49 g |

| | | |
|---|---|---|
| Filter Soak: | 0.5% PEI | 10 grams/litre |

Another assay using a different source of transporter is obtained from D'Amato, R. J., Largent, B. L., Snowman, A. M., and Snyder, S. H. Selective Labeling of Serotonin Uptake Sites in Rat Brain by [³H] Citalopram Contrasted to Labeling of Multiple Sites by [³H] Imipramine. Jrn. Pharmacol. & Exp. Ther. 242: 364-371 (1987) with modifications.

Tissue Preparation:

1. Human Platelet enriched plasma was obtained from commercial sources. Platelets were centrifuged at 1000× g for 10 minutes at room temperature. Supernatant was discarded into a bleach-containing beaker.

2. Pellet were resuspended in 20 volumes of assay buffer (50 mM TRIS-HCl containing 120 mM NaCl and 5 mM KCl (pH 7.4 at 25° C.)) and homogenized with a polytron at setting 5 for 10 seconds. A small aliquot was removed for protein determination using standard methods.

3. The tissue homogenate was centrifuged at 48,400× g at 4° C. for 10 minutes. Supernatant was discarded.

4. The pellet was resuspended to 5 mg protein/ml in assay buffer and frozen at −40C until needed.

5. On the day of the assay, a tissue aliquot was diluted and mixed by using a polytron (setting 5; approximately 10 seconds), and the pellet was resuspended in 50 mM TRIS-HCl containing 120 mM NaCl and 5 mM KCl (pH 7.4 at 25° C.) to an initial concentration of 0.5 protein/ml, such that the final tissue concentration was 0.4 mg/ml or 0.1 mg/tube.

Binding Reaction

1. Each tube received the following components:

25 ul drug or vehicle

25 ul [³H]-Citalopram

200 ul tissue suspension

2. The binding reaction was initiated with the addition of tissue, and incubated for 60 minutes at 25° C.

3. The binding reaction was terminated by rapid vacuum filtration of tube contents onto presoaked (0.5% PEI for at least 1 hour) GF/C filters.

4. The tubes were rinsed once with ice cold 50 mM NaCl, then the filters were rapidly rinsed with approximately 5 ml/tube of the same ice cold wash buffer.

5. Radioactivity trapped on the filters was assessed using a TopCount Scintillation Counter. Filters were dried overnight or in an oven, then wells of the plate were filled with scintillation fluid, and the plate was allowed to sit for one hour prior to counting.

Materials and Reagents

1. [³H]-Citalopram was diluted in 50 mM TRIS-HCl containing 120 mM NaCl and 5 mM KCl (pH 7.4 at 25° C.) to a concentration of 7 nM, such that the final radioligand concentration in the assay was 0.7 nM.

2. Non-specific binding was defined as that remaining in the presence of 10 μM.

Imipramine, which was solublized in dH₂O.

3. The reference compound was imipramine run at final concentrations of: (imipramine was solublized in dH₂O) 1×10⁻¹⁰, 2×10⁻¹⁰, 5×10⁻¹⁰, 1×10⁻⁹, 2×10⁻⁹, 5×10⁻⁹, 1×10⁻⁸, 2×10⁻⁸, 5×10⁻⁸, 1×10⁻⁷, 2×10⁻⁷, 5×10⁻⁷ M.

4. The positive control was imipramine run at final concentrations of 1×10⁻⁹, 1×10⁻⁷, and 1×10⁻⁵ M.

5. The K_{d} of the serotonin transporter for [³H]-Citalopram was 3 nM.

| Buffers: | | MW (g/mole) |
|---|---|---|
| Assay Buffer: | 50 mM TRIS-HCl (pH 7.4) | 121.0 |
| | 120 mM NaCl | 58.44 |
| | 5 mM KCl | 74.55 |

### EXAMPLE 5

### Assay for Norepinephrine Transporter Activity

Cocaine (and other addictive chemicals or other compounds added to the database) were prepared as described in Example 1 and tested for activity at the norepinephrine transporter according to the following protocol. One ordinarily skilled in the art would recognize that other assay protocols or modifications to the protocol below could provide the same type of information required regarding determination of the activity of addictive or other chemicals at the norepinephrine transporter molecular target.

The assay is derived from Raisman, R., Sette, M., Pimoule, C., et.al. High Affinity [3H] Desipramine Binding in the Peripheral and Central Nervous System: A Specific Site Associated with the Neuronal Uptake of Noradrenaline. Eur. Jrnl. Pharmacol. 78: 345-351 (1982) with modifications.

Tissue Preparation

1. Brains from male Sprague Dawley rats were removed shortly following decapitation and placed in ice cold 50 mM TRIS HCl pH 7.4 with 5 mM KCl and 120 mM NaCl. The forebrain region was isolated.

2. The forebrain was homogenized in 50 volumes (weight/volume) of 50 mM TRIS HCl pH 7.4 with 5 mM KCl and 120 mM NaCl using a polytron at setting 5 for approximately 20 seconds.

3. The homogenate was centrifuged at 48,400× g for 10 minutes at 4° C. Supernatant was discarded.

4. Steps 2 and 3 were repeated for three more washes.

5. The pellet was stored on ice until needed for the binding assay.

6. Using a Polytron (setting 5; approximately 10 seconds) the pellet was resuspended to an initial concentration of 30 mg (original wet weight)/ml in 50 mM TRIS HCl pH 7.4 with 5 mM KCl and 300 mM NaCl, such that the final tissue concentration was 24 mg/ml or 6 mg tissue/tube.

Binding Reactions

1. Each tube received the following components:

25 ul drug or vehicle

25 ul [³H]-Nisoxetine

200 ul tissue suspension.

2. The binding reaction was initiated with the addition of tissue, and incubated on ice (0° C.) for 4 hours.

3. The binding reaction was terminated by rapid filtration of tube contents onto untreated GF/B filters (Betaplate).

4. The assay tubes were rinsed once with ice cold 150 mM NaCl, then the filters were rapidly rinsed with 6×1 ml/tube of the same wash buffer.

5. Radioactivity trapped onto the filters was assessed using liquid scintillation spectrophotometry after soaking the filters for at least 1 hour in scintillation cocktail.

Material and Reagents

1. [³H]-Nisoxetine was diluted to a concentration of 10 nM in 50 mM TRIS HCl pH 7.4 with 5 mM KCl and 300 mM NaCl. Thus, the final ligand concentration was 1.0 nM.

2. Non specific binding was defined as that remaining in the presence of 1×10⁻⁶M desipramine (MW=302.8).

3. The reference compound for the assay was desipramine, imipramine, amitriptyline or nisoxetine. Desipramine was run, preferentially, at following final concentrations: 5×10⁻¹¹, 1×10⁻¹⁰, 2×10⁻¹⁰, 5×10⁻¹⁰, 1×10⁻⁹, 2×10⁻⁹, 5×10⁻⁹, 1×10⁻⁸, 2×10⁻⁸, 5×10⁻⁸, 1×10⁻⁷, 2×10⁻⁷ M.

4. The positive control was any of the above compounds (preferably desipramine) run at the final concentrations of: 1×10⁻⁹, 5×10⁻⁹, 2×10⁻⁸ M.

5. The K_{d} for the transporter is 0.9 nM.

Both desipramine and imipramine were dissolved in water. Water was added to the desired concentration and the solution was sonicated for approximately 10 minutes.

| BUFFERS: | | MW (g/mole) |
|---|---|---|
| Tissue Prep Buffer: | 50 mM Tris-HCl pH 7.4 | 6.06 g/L |
| | 5 mM KCl | 0.38 g/L |
| | 120 mM NaCl | 7.02 g/L |
| Incubation Buffer: | 500 ml Tissue Prep buffer plus 5.5 g NaCl for | |
| | 300 mM NaCl | |
| Wash Buffer: | 150 mM NaCl | 9.0 g/L |

### EXAMPLE 6

### Assay for Sigma 1 Receptor Activity

Cocaine (and other addictive chemicals or other compounds added to the database) were prepared as described in Example 1 and tested for activity at the sigma 1 receptor according to the following protocol. One ordinarily skilled in the art would recognize that other assay protocols or modifications to the protocol below could provide the same type of information required regarding determination of the activity of addictive or other chemicals at the sigma 1 receptor molecular target.

The assay is derived from Chaki, S., Tanaka, M., Muramatsu, M. and Otomo, S., NE-100, a novel potent σ ligand, preferentially binds to σ₁ binding sites in guinea pig brain. Eur. J. Pharmacol. 251 R1-R2 (1994).

Tissue Preparation

1. Fresh guinea pig whole brain was obtained following decapitation and exposure to 100% CO₂ gas.

2. Using a Polytron (setting 5; approximately 30 seconds), whole brains were homogenized in about 10 volumes of ice cold 50 mM Tris, pH 7.4.

3. The homogenate was centrifuged at about 48,400× g for 10 minutes at 4° C.

4. The resulting pellet was resuspended in fresh buffer and recentrifuged at 48,400× g for 10 minutes at 4° C. Supernatant was discarded.

5. Using a Polytron (setting 5; approximately 10 sec.) the pellet was resuspended to an initial concentration of 25 mg tissue (original wet weight)/ml in 50 mM Tris-HCl (pH 7.4 at 25° C.) such that the final concentration in the assay was 20 mg/ml or 5 mg tissue/tube.

Binding Reaction

1. Each tube received the following components:

25 ul drug or vehicle

25 ul [³H]-(+)-Pentazocine

200 ul tissue suspension

2. The binding reaction was initiated with the addition of tissue and incubated for 120 minutes at 25° C.

3. The binding reaction was terminated by rapid vacuum filtration of the assay tube contents onto TopCount GF/B filters (filters were pretreated with 0.1% PEI for 30 min.).

4. The assay tubes were rinsed 5 times with ice cold normal saline.

5. Radioactivity trapped onto the filters was assessed using a TopCount Scintillation Counter after soaking the filters for at least one hour in scintillation cocktail.

Material and Reagents

1. [³H]-(+)-Pentazocine was diluted in 50 mM Tris-HCl (pH 7.4 at 25° C.) to a concentration of 20 nM such that the final radioligand concentration in the assay was 2.0 nM.

2. Non-specific binding was defined as that remaining in the presence of 1×10⁻⁶M haloperidol (MW=375.88). (Made in 100% EtOH to initial concentration of 1×10⁻³M).

3. The reference compound was haloperidol, run at the following final concentrations: 2×10⁻¹¹, 5×10⁻¹¹, 1×10⁻¹⁰, 2×10⁻¹⁰, 5×10⁻¹⁰, 1×10⁻⁹, 2×10⁻⁹, 5×10⁻⁹, 1×10⁻⁸, 2×10⁻⁸, 5×10⁻⁸, 1×10⁻⁷M.

4. The positive control was haloperidol run at final concentrations of 2×10⁻⁹, 2×10⁻⁸, 1×10⁻⁷M.

6. The K_{d} of the receptor for [³H]-(+)-Pentazocine was 11 nM.

### EXAMPLE 7

### Assay for Serotonin 5HT-3 Receptor Activity

Cocaine (and other addictive chemicals or other compounds added to the database) were prepared as described in Example 1 and tested for activity at the serotonin 5HT-3 receptor according to the following protocol. One ordinarily skilled in the art would recognize that other assay protocols or modifications to the protocol below could provide the same type of information required regarding determination of the activity of addictive or other chemicals at the serotonin 5HT-3 receptor molecular target.

The assay is derived from Lummis, S. C. R., Kilpatrick, G. J. Characterization of 5HT₃ Receptors in Intact N1E-115 Neuroblastoma Cells. European Journal Pharmacology. 189: 223-227 (1990) with modifications.

Tissue Preparation

1. N1E-115 mouse neuroblastoma cells were grown up in the tissue culture facility in T-150 flasks to sub-confluency using standard culture procedures.

2. Flasks were shaken to remove the cells from the sides of the flasks. The sides of the flasks were rinsed with the media. The culture media containing the cells was then removed from the flask and transferred to 50 ml conical tubes.

3. The media containing the cells was centrifuged in a Sorvall tabletop centrifuge (1500 rpm, 10° C.) for 10 minutes.

4. The pellets were gently resuspended in approximately 10 mls of 20 mM HEPES containing 150 mM NaCl (pH 7.4 at 25° C.).

5. The cells were homogenized using a polytron (setting 5; approximately 10 seconds). This homogenate was centrifuged at 48,400× g at 4° C. for 10 minutes. Supernatant was discarded.

6. The pellet was resuspended and a protein determination was performed using standard methods.

7. Using a polytron (setting 5; approximately 10 seconds), cells were resuspended in approximately 10 ml of 20 mM HEPES buffer containing 150 mM NaCl (pH 7.4 at 250 C) and centrifuged again as on Step 5. Supernatant was discarded.

8. This membrane preparation was diluted in 20 mM HEPES (pH 7.4 at 25° C.) containing 150 mM NaCl to 325 ug protein/ml, so that each tube received 100 ug of protein, or 250 ug/ml.

Binding Reaction

1. Each tube received the following components:

50 ul drug or vehicle

50 ul [³H]-GR65630

400 ul tissue suspension

2. The binding reaction was initiated by addition of tissue and incubated at 25° C. for 60 minutes.

3. The binding reaction was terminated by rapid vacuum filtration of the assay tube contents onto untreated GF/B filters.

4. The assay tubes were rinsed 5 times with ice cold 50 mM HEPES containing 150 mM NaCl (pH 7.4 at 25° C.).

5. Radioactivity trapped on the filters was assessed using liquid scintillation spectrophotometry after soaking the filters for at least three hours in scintillation cocktail.

Material and Reagents

1. [³H]-GR65630 was diluted in 50 mM HEPES containing 150 mM NaCl (pH 7.4 at 25° C.) to a concentration of 3.5 nM such that the final concentration was 0.35 nM.

2. Nonspecific binding was defined as that remaining in the presence of 1 uM MDL72222. (NOTE: MDL72222 must first be dissolved in 100% DMSO).

3. The reference compound was MDL72222 run at the following final concentrations: 5.0E-11; 1.0E-10; 2.0E-10; 5.0E-10; 1.0E-9; 2.0E-9; 5.0E-9; 1.0E-8; 2.0E-8; 5.0E-8; 1.0E-7; 2.0E-7 M.

4. The positive control was MDL72222, run at the following final concentrations: 1×10⁻⁷, 3×10⁻⁸, 1×10⁻⁸ M.

5. The K_{D} of the 5HT₃ receptor for [³H]-GR65630 was 0.35 nM.

| BUFFERS | | Molecular Weight |
|---|---|---|
| Assay | 20 mM HEPES pH 7.4 | 238.31 |
| | 150 mM NaCl | 58.44 |
| Wash | 150 mM NaCl | 58.44 |

### EXAMPLE 8

### Assay Data Handling and Data Analysis

For each assay, cocaine (or other addictive chemicals or other compounds included in the database) were tested in Pass 1 at 10⁻⁵ M. Data were calculated as the percent inhibition of specific binding at each concentration. All datapoints, rather than means of individual points, were included in the system database. For those assays where duplicate datapoints were associated with a co-variance of >20 percent, the cocaine sample (or other compound) was retested. Statistical routines (e.g., Dixon test) were applied to eliminate outlying points from QA/QC data, such as individual points in a reference curve. The determination of the percent inhibition of the cocaine samples was performed using computer programs that have been developed and validated by NovaScreen Biosciences Corporation specifically for this purpose. Data from counters or detectors were directed automatically to Excel under Windows NT platform. All pertinent calculations were performed automatically on local area PC-based workstations, and compiled into a Microsoft Access or Oracle Database.

The Pass 1 percent inhibition threshold was set at 30% for all 131 assays. For all assays for which cocaine demonstrated less than 30% inhibition at 10⁻⁵ M, the percent inhibition values were entered in the system database but according to the threshold criteria, no relevant activity was detected, essentially establishing a set of negative data. No further testing of cocaine was performed for these assays or molecular targets. All assays in which cocaine demonstrated average percent inhibition greater than or equal to 30% in Pass 1 were repeated at the Pass 2 testing protocol (see Example 1) for cocaine at three concentrations ranging from 10⁻⁵M to 10⁻⁹M. Of the 131 assays, 18 met the threshold to go to Pass 2 testing.

The Pass 2 percent inhibition threshold was set at 75% at 10⁻⁵ M for all remaining assays. For all assays for which cocaine demonstrated less than 75% inhibition at 10⁻⁵ M in both Pass 1 and Pass 2, the percent inhibition values for Pass 2 at all three test concentrations were also entered in the system database. No further testing of cocaine was performed for these assays or molecular targets (except for those demonstrating greater than 50% inhibition, see below). All assays in which cocaine demonstrated average percent inhibition greater than or equal to 75% in Pass 1 and Pass 2 at 10⁻⁵ M were repeated at the Pass 3 testing protocol (see Example 1) for cocaine at nine concentrations, with the specific concentration range selected for each assay, preferably such that the 50% inhibitory concentration (IC₅₀) was bracketed with at least three concentrations on the slope of the curve. Of the 131 assays, two (serotonin transporter and dopamine transporter) met the 75% inhibition (highest potency) threshold criteria to go to Pass 3 testing.

The Pass 3 data, designed to establish the relative and absolute (quantitative) potency of cocaine at the specific targets, were entered into the system database for each of the two above-threshold assays. An IC₅₀ value and Ki were calculated using XLFit (IDBS) and entered into the system database.

After analysis of the Pass 1 to Pass 3 datasets, one additional threshold range was established to designate assays or targets in which cocaine demonstrated marginal activity that may or may not be physiologically relevant. This threshold range was established at >50% and <75% inhibition at 10⁻⁵ M for the Pass 1 or Pass 2 screening data. Three additional targets, which were the Sigma-1 receptor, Serotonin 5HT3 receptor, and Norepinephrine transporter met this additional threshold criterion. For each of these three additional molecular targets, cocaine was tested in these assays according to Pass 3 protocols described above and an IC₅₀ and Kᵢ were determined for cocaine for each target using the Pass 3 data, as shown in Table 2.

### EXAMPLE 9

### Cocaine Activity Database Annotations

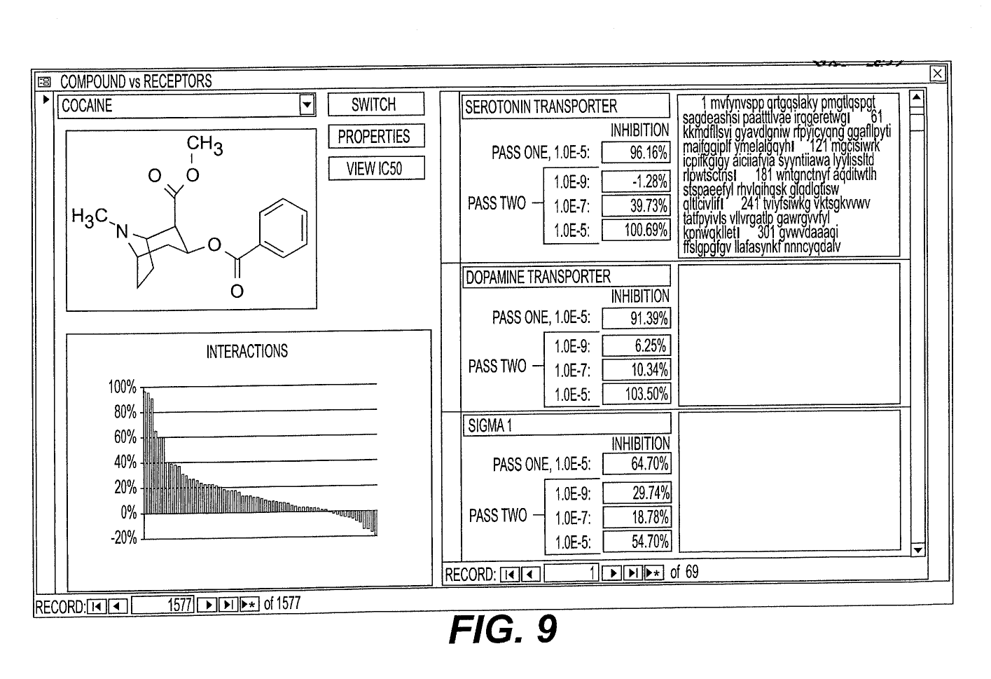

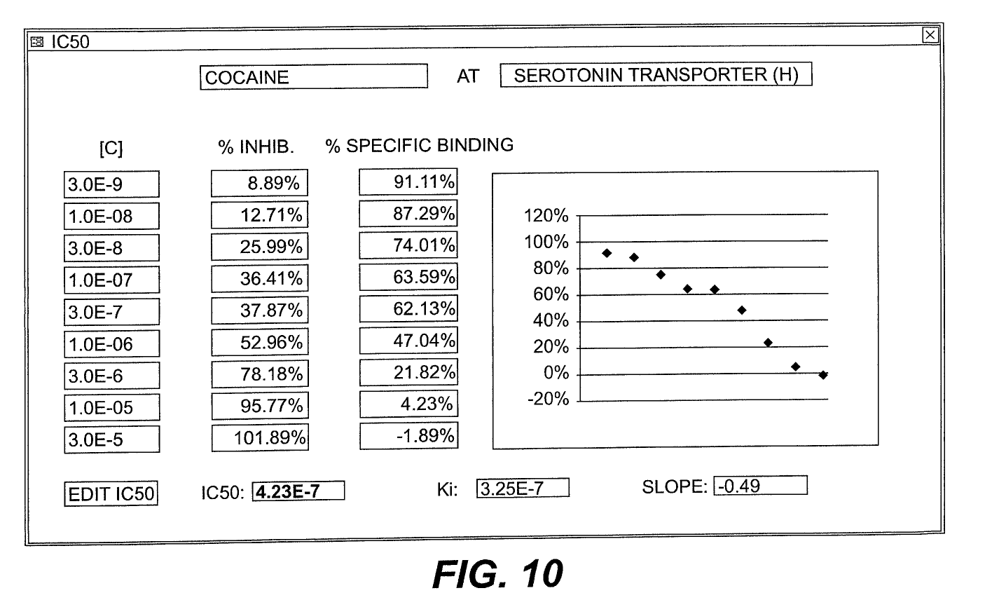

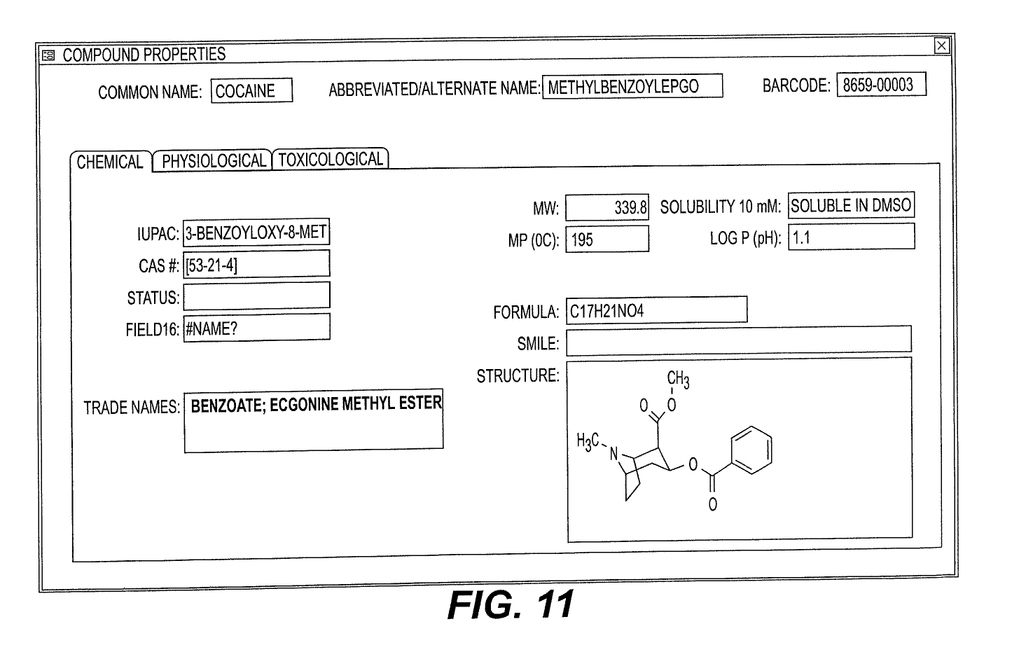

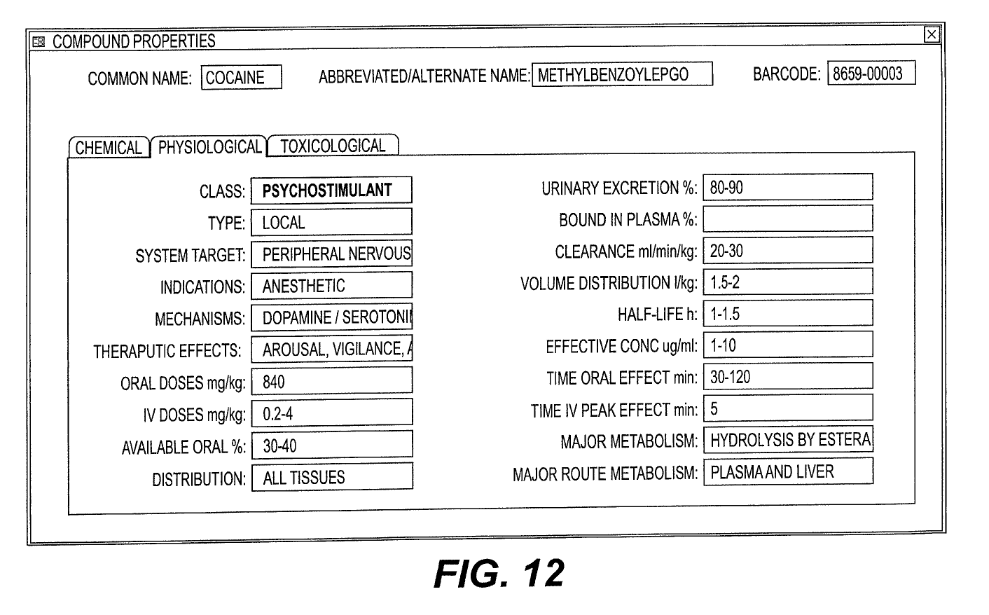

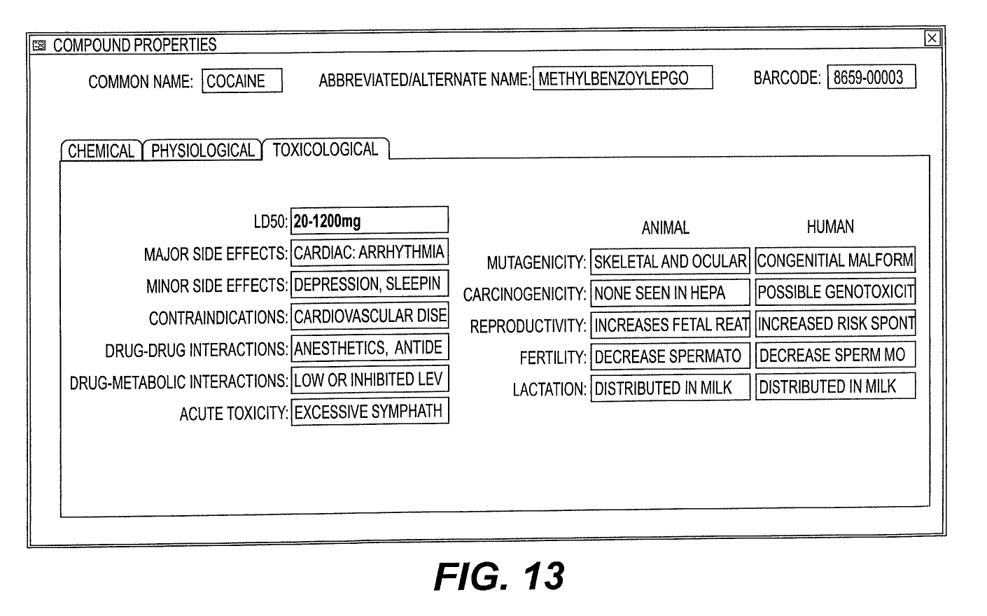

The in vitro reactivity information obtained from testing cocaine (and other compounds in the database), as described above and shown in Tables 1 and 2, was entered into the compound activity table (cocaine field) of the database. FIG. 9 shows a database screen shot containing Pass 1 and Pass 2 activity data. FIG. 10 shows a database screen shot containing IC₅₀ and Kᵢ data. Certain chemoinformatic information for cocaine including the 2D structure, solubility, molecular weight, and LogP value, was annotated into the system database. FIG. 11 depicts one screen shot from the database of the Compound Properties—Chemical (chemoinformatics) table (cocaine field). Data regarding the in vivo properties of cocaine, derived from the scientific literature and other sources, were also entered into the database. FIG. 12 depicts one screen shot from the database of the Compound Properties—Physiological table (cocaine field). FIG. 13 depicts one screen shot from the database of the Compound Properties—Toxicological table (cocaine field). Other chemoinformatic and available in vivo and in vitro activity data on, or properties of, cocaine (and other compounds in the database) are entered in other tables and fields in the database. These physiological, toxicological, and other data previously known for cocaine, together with the in vitro biological activity determined according to Examples 1-8 and the annotations in the chemoinformatics fields, were analyzed using the database and data mining methods to draw conclusions regarding compositions of targets relevant to cocaine's activity and to identify approaches to treatment of cocaine addiction.

### Example 10

### Molecular Target Database Annotations

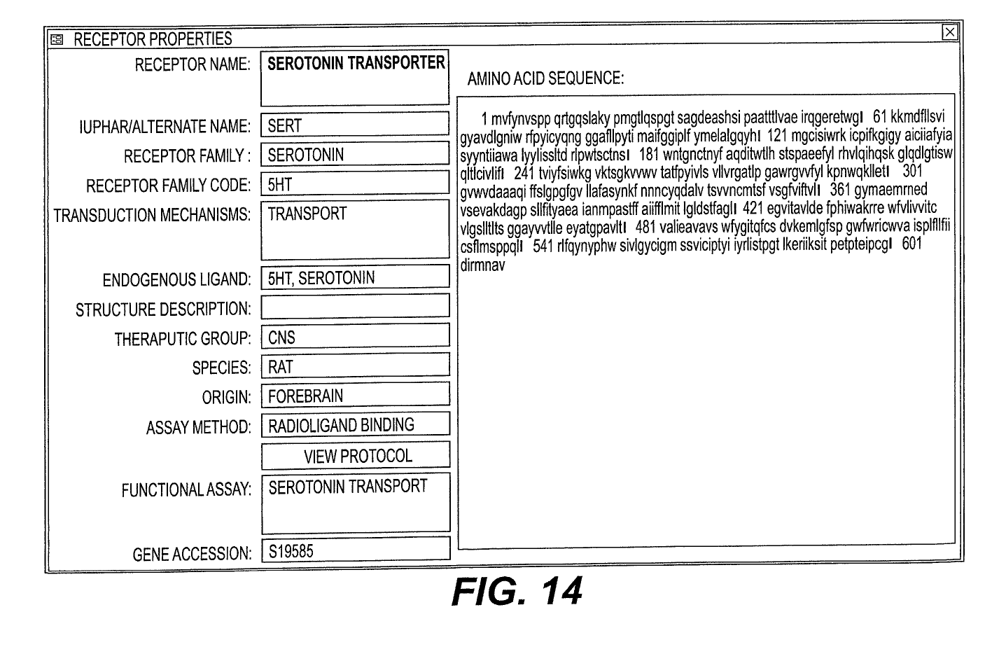

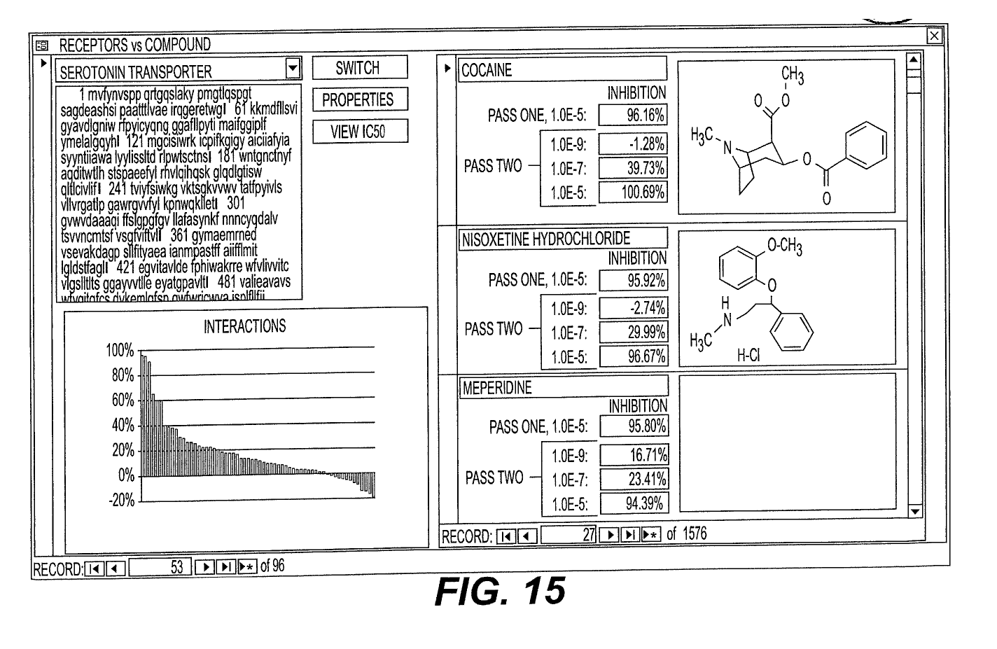

Another important content of the system database is bioinformatic information associated with the molecular targets, such as peptide (amino acid) sequences of the receptors, transporters, ion channels, and enzymes, and known biochemical and associated physiological functions of these molecular targets. Bioinformatic annotations include nomenclature, protein family, signal transduction linkage, endogenous ligand or substrate, gene accession reference number, linkage to genetic sequence and gene expression databases, and assay information such as the preferred protocol, source of molecular target, etc. FIG. 14 is one screen shot from the database (serotonin transporter field) showing representative bioinformatic annotations as an example. Fields for each assay or molecular target are included in the system database. All information is stored in, and separated by, linked tables in the database to enable data mining and data comparison. FIG. 15 is a database screen capture, used herein as an example to depict that the molecular target data annotations are extended beyond the peptide sequences (as found in a conventional bioinformatic database) to include the overall chemical reactivity profiles and potency of the interactions between the target and different chemicals.

### EXAMPLE 11

### Relational Database Component Interrogation

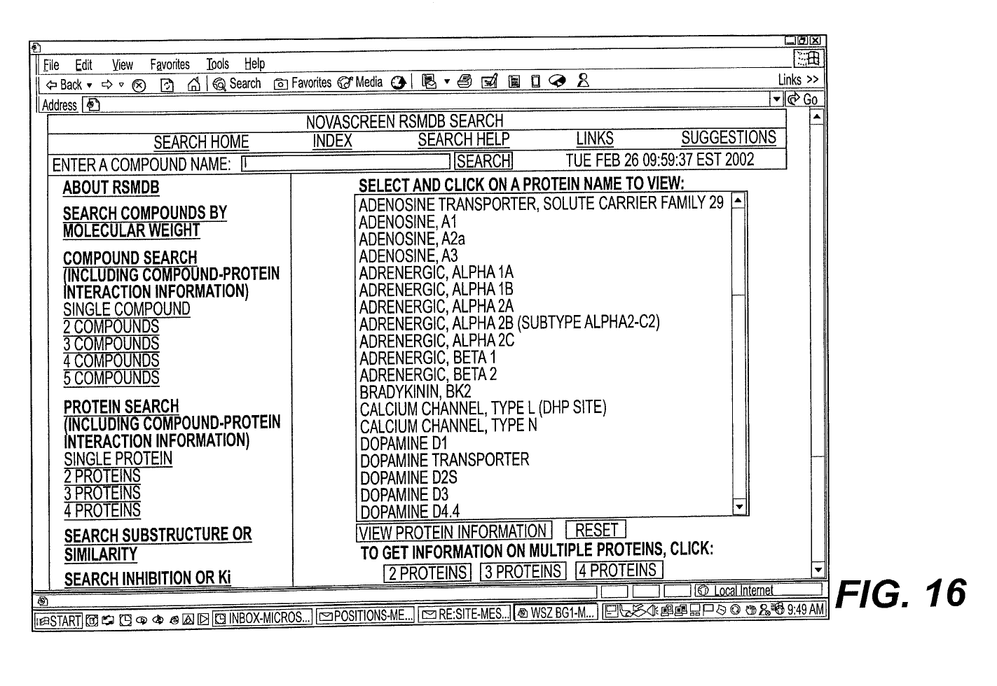

Switches were built into the database query interface so that comparisons and analyses can be made between a selected target and any single compound or set or subset of the different chemicals (see FIG. 16 for one database screen shot as an example) or between a selected chemical and any single, set, or subset of different targets (see FIG. 9 for one database screen shot as an example). The executable “Switch” as shown in the examples will extend or switch the interface to show either comparisons between selected compounds vs. receptors (or other targets) or selected receptors (or other targets) vs. compounds. The executable “Properties” switch (from the entry point of a chemical) will extend or switch the interface from the selected chemical vs. in vitro activity component of the database for one or multiple molecular targets in the database (e.g., as shown in FIG. 9) to the chemoinformatic, physiological, toxicological, and related annotations for the selected chemical (for example, as shown in FIGS. 11-13). The executable “Properties” switch (from the entry point of a molecular target) will extend or switch the interface from the selected target vs. in vitro activity component of the database for one or a multitude of the chemicals in the database (e.g., as shown in FIG. 15) to the bioinformatic annotations of the selected receptor, ion channel, enzyme, transporter, or other molecular target (for example, as shown in FIG. 14).

### EXAMPLE 12

### Data Mining and Identification of Key Targets Associated with Cocaine Action and Properties

FIG. 16 shows an intranet web page, which depicts a screen shot for an interface to the system database that can be used to interrogate information housed in the database. In order to identify the relevant biological targets related to a given chemical dependency (such as cocaine addiction), the chemical (such as cocaine) was chosen as a “single chemical”. The biological profiles (Pass 1 in vitro screening data) for cocaine were analyzed for activity against molecular targets (that is, in the respective assays) that fell within the threshold activity ranges established for highest biological activity and for marginal but potentially still physiologically relevant biological activity.

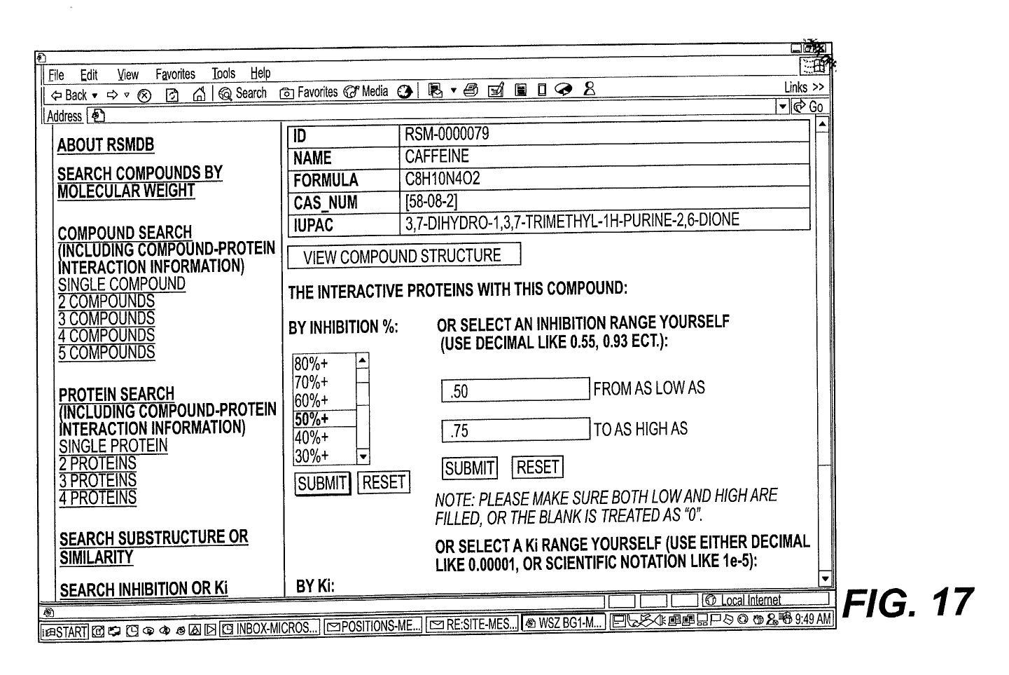

FIG. 17 is another screen shot from the interface to the system database showing that various threshold ranges for biological activity (at, for example, a concentration of the chemical of 10⁻⁵M) can be selected and implemented according to the objectives for establishing the set of relevant biological targets for the selected chemical. Once such a profile of relevant molecular targets for cocaine was established, the validity of the initial threshold determinations was confirmed by a comparison in the database between the Pass 1 percent inhibition and the Kᵢ determined from the Pass 3 screening data, which provided more quantitative information regarding the activity of cocaine at the identified molecular targets.

Following identification of the key targets for cocaine activity, an analysis was made between the bioinformatic annotations for the selected targets and the physiological, toxicological, and other in vivo or in vitro properties of cocaine. Furthermore, the database and query interface to the database allow for the analysis of activities and comparison of multiple relevant molecular targets at one time, as shown in FIG. 16. These analyses were used as the guide for the selection of molecular targets critical for identifying and designing medications potentially useful in treating cocaine addiction or other chemical dependency.

### EXAMPLE 13

### Expansion of In Vitro Activity Screening Dataset to Other Known Addictive Substances

The cocaine activity profile database established as described in Examples 1-8 above and the chemoinformatic database annotations described in Example 9 above provide important information useful for deriving methods to treat addiction to cocaine. This cocaine-related database, while a multidimensional database in that it contains (1) chemical/compound information, (2) multiple molecular targets and target annotations, (3) in vitro activity data between a compound (cocaine) and multiple targets, and (4) biological/in vivo information on the compound and on that associated with the targets, is only one-dimensional with respect to the chemical compound component. The utility of such a multidimensional database can be expanded by the inclusion of additional chemical compounds, the testing of these compounds for activity at a multitude of molecular targets, and entering the resulting data as well as annotations on the additional compounds to the database.

One expansion of the compound set that is relevant to cocaine addiction and substance abuse or chemical dependency in general is to add known addictive substances to the database. Chemicals that can be added to the dataset to create an addiction database include those designated as controlled substances by the Drug Enforcement Agency (DEA) of the U.S. Government. Such chemicals are designated under the classifications of Schedules I-V, with each category generally representing a relative increase (I being the highest) of addictive liability. Chemicals designated as Schedule I-V controlled substances by the DEA are listed in Table 3. Such chemicals, most having documented in vivo biological effects, are useful additions to the multidimensional database relating in vivo data and in vitro activities between compounds and targets comprising a portion of this invention.

TABLE 3

| | |
|---|---|
| Class of Compounds | Compound Type |
| Chemical Name | Trade/Other Names |

Schedule I (all nonresearch use is illegal)

| | | |
|---|---|---|
| flunitrazepam | | |
| flunitrazepam | | Rohypnol, Ecstasy |
| Narcotics | | |
| heroin | | |
| Hallucinogens | | |
| LSD | | |
| MDA | hallucinogen | |
| STP | hallucinogen | |
| DMT | hallucinogen | |
| DET | hallucinogen | |
| mescaline | hallucinogen | |
| peyote | hallucinogen | |
| bufotenine | hallucinogen | |
| ibogaine | hallucinogen | |
| psilocybin | hallucinogen | |
| phencyclidine/PCP | hallucinogen | |
| marijuana | | |
| marijuana | | |
| methaqualone | | |
| methaqualone | | |

Schedule II (no telephone prescriptions, no refills)

| | | |
|---|---|---|
| Opioids | | |
| opium | | |
| morphine | opium alkaloids | |
| hydromorphone | opium alkaloids | Dilaudid |
| oxymorphone | opium alkaloids | Numorphan |
| oxycodone | opium alkaloids | Percodan, Percocet, |
| | | Roxicodone |
| levomethadyl | synthetic drugs | Orlaam; |
| | | dihydroxycodonone |
| meperidine | synthetic drugs | Demerol |
| methadone | synthetic drugs | |
| levorphanol | synthetic drugs | Levo-Dromoran |
| fentanyl | synthetic drugs | Sublimaze, Duragesic, |
| | | Actiq |
| alphaprodine | synthetic drugs | |
| alfentanil | synthetic drugs | Alfenta |
| sulfentanil synthetic drugs | Sufenta | |
| remifentanil | synthetic drugs | Ultiva |
| Stimulants | | |
| cocaine | | |
| amphetamine | | |
| amphetamine complex | | Biphetamine |
| dextroamphetamine | | Dexedrine |
| methamphetamine | | Desoxyn |
| phenmetrazine | | Preludin |
| methylphenidate | | Ritalin |
| Depressants | | |
| amobarbital | | Amytal |
| pentobarbital | | Nembutal |
| secobarbital | | Seconal |
| (mixtures) | | Tuinal |

Schedule III (prescription rewritten after 6 mos/5 refills)

| | |
|---|---|
| Opioids | |
| codeine | |
| hydrocodone | Hycodan, Vicodin, |
| | Lortab |
| opium | paregonc |
| Stimulants | |
| benzphetamine | Didrex |
| phendimetrazine | Plegine |
| Depressants | |
| butabarbitol | Butisol |
| thiopental | Pentothal |
| Cannabinoids | |
| dronabinol | Marinol |
| Anabolic steroids | |
| fluoxymesterone | Halotestin |
| methyltestosterone | Android, Testred |
| nandrolone decanoate | Dec-Durabolin |
| nandrolone phenpropionate | Durabolin |
| oxandrolone | Oxandrin |
| oxymetholone | Androl-50 |
| stanazolol | Winstrol |
| testolactone | Teslac |
| testosterone | |

Schedule IV (prescription rewritten after 6 mos/5 refills)

| | | |
|---|---|---|
| Opioids | | |
| butorphanol | | Stadol |
| difenoxin | | Motophen |
| pentazocine | | Taiwin |
| propoxyphene | | Darvon |
| Stimulants | | |
| diethyipropion | | Tenuate |
| mazindol | | Sanorex |
| phentermine | | Jonamin |
| pemoline | | Cylert |
| sibutramine | | Merida |
| Depressants | | |
| alprazolam | benzodiazepine | Xanax |
| chlordiazepoxide | benzodiazepine | Librium |
| clonazepam | benzodiazepine | Kionopin |
| clorazepate | benzodiazepine | Tranxene |
| diazepam | benzodiazepine | Valium |
| estazolam | benzodiazepine | ProSom |
| flurazepam | benzodiazepine | Dalmane |
| halazepam | benzodiazepine | Paxipam |
| lorazepam | benzodiazepine | Ativan |
| midazolam | benzodiazepine | Versed |
| oxazepam | benzodiazepine | Serax |
| prazepam | benzodiazepine | Centrax |
| quazepam | benzodiazepine | Doral |
| temazepam | benzodiazepine | Restoril |
| triazolam | benzodiazepine | Halcion |
| chloral hydrate | | |
| ethchlorvynol | | Placidyl |
| meprobamate | | Equanil, Miltown |
| mephobarbital | | Mebaral |
| methohexital | | Brevital |
| paraldehyde | | |
| phenobarbital | | |
| zaleplon | | Sonata |
| zolpidem | | Ambien |

Schedule V

| | |
|---|---|
| Opioids | |
| buprenorphine | Buprenex |
| diphenoxylate | Lomotil |
| codeine | |
| dihydrocodeine | |

Chemicals selected from the list in Table 3 or otherwise added to the database are assembled into solutions for screening repositories, each comprising one of the chemicals and in concentrations and procedures as described in Example 1 for cocaine. Each chemical is tested in assays selected from those listed in Table 1, including those described in Examples 3-7, and as generally described in Example 2, based on a scheme identified as Passes 1-3 or by other procedures to establish whether various thresholds of activity are reached for each compound at each target and/or to determine quantitative values of potency for each compound active at a target.

In vitro screening data determined according to the above procedures is then analyzed as described in Example 8 above and entered into the multidimensional database. Chemoinformatic annotations for each such compound are entered into the database, as described in Example 9.

All patent, patent applications, and publications mentioned are incorporated by reference in their entirety into this application.

### REFERENCES:

Ali, S. f. (1998) Ann. New. York Acad. Sci. 844, 122

Chait, I. D.; Uhlenhuth, E. H.; and Johanson, C. E. (1987) J. Pharmacol. Exp. Ther. 242 777.

Giros, B.; Jaber, M.; Jones, S. R.; Wightman, R. M.; Caron, M. G. (1996) Natural 379 606

Gorelick, D. A. (1998) Adv. Pharmacol. 42 995.

Herz, A. (1998) Can. J Phys. Pharmacol. 76, 252

Klein, M. (1998) Ann. New York Acad. Sci. 844 75.

Koob, G. F. (1998) Adv. Pharmacol. 42, 969.

Mash, D. C. and Staley J. K. (1998) in Neurochemistry of Drug Abuse, Drug Abuse HandBook, (Karch, S. B. ed) CRC press, pp.395⁺ and references therein.

Methews, J. C. and Collins, A. (1983) Biochem. Pharmacol. 32, 455.

Rocha, B. A. et al (1998) Nat. Neurosci. 1, 132

Rocha, B. A. et al (1998) Nature 393, 175

Self, D. W. and Nestler, E. J. (1995) Rev. neurosci. 18. 463

Self, D. W. et al (1996) Science 271 1586.

Shuster, L. (1991) in Cocaine: Pharmacology, Physiology and Clinical Strategies (Lakoski, J. M., Galloway, M. P., and White, F. J.; eds) CRC press, pp.1+ and references therein.

Smith, M. P.; Hoepping, A.; Johnson, K. M.; Trzcinska, M.; and Kozikowski, P. (1999) Drug Disc. Tech. 7, 322.

Sora, I. et al (1998) Proc. Natl. Acad. Sci. U.S.A. 95 7699

Sora, I et al (2001), Proc. Natl. Acad. Sci. U.S.A. 98 5300

The foregoing description of embodiments of the present invention provides an exemplary illustration and description, but is not intended to be exhaustive or to limit the invention to the precise form disclosed. Modifications and variations are possible in light of the above teachings or may be acquired from practice of the invention.

We claim:

1. A method for determining a biological activity profile for an abused or addictive substance, comprising:
(a) selecting a panel of molecular targets;
(b) defining a pharmacological activity profile for the abused substance by;
(i) exposing the abused substance to each of the molecular targets in the panel; and
(ii) measuring the ability of the abused or addictive substance to interact with each of the molecular targets; and
(c) determining the biological activity profile by identifying in the pharmacological activity profile a subset of the molecular targets whose activity is effected by the abused substance.

2. A method for determining a set of one or more molecular targets for developing a treatment for abuse of, or addiction to, a substance, comprising:
(a) determining a biological activity profile according to the method of claim 1 for the abused or addictive substance; and
(b) defining the set of molecular targets by identifying those targets in the biological activity profile wherein the interaction between the abused or addictive substance and the molecular target exceeds a threshold level.

3. The method of claim 2, further comprising defining the set of molecular targets by utilizing information relating the pathology associated with abuse or addiction to the substance to the interaction of the substance with different molecular targets.

4. The method of claim 3, wherein the information utilized includes either or both of information concerning at least one positive effect of the abused or addictive substance and information concerning at least one negative effect of the abused or addictive substance.

5. The method of claim 4, wherein the information exists in a relational database.

6. A method for identifying at least one chemical compound to treat abuse or addiction of a substance, comprising:
(a) determining a set of molecular targets according to the method of claim 2;
(b) providing a database of chemical compounds containing records corresponding to a plurality of chemical compounds, wherein said records include data on the interaction of the chemical compounds with a plurality of molecular targets; and
(c) selecting one or more chemical compounds from the database, wherein the selected chemical compound or compounds interact with the molecular targets in a manner substantially the same as the abused or addictive substance.

7. The method according to claim 6, wherein the set of molecular targets is determined according to the method of claim 3.

8. The method according to claim 6, wherein the database of chemical compounds is a relational database.

9. The method of claim 6, wherein one or more assays are used to define the pharmacological activity profile.

10. The method of claim 9, wherein each assay comprises a buffer solution, a cell or tissue preparation containing a molecular target, and a labeled compound that interacts with the molecular target.

11. The method of claim 10, wherein the molecular target is a receptor, transporter, ion channel, or enzyme.

12. The method of claim 10, wherein the wherein the molecular target is from animal tissue, human tissue, or cultured cells.

13. The method of claim 12, wherein the cultured cells natively express the molecular target.

14. The method of claim 12, wherein the cultured cells express a recombinant nucleic acid encoding the molecular target.

15. The method of claim 10, wherein the molecular target is a crude preparation, partially purified, or highly purified.

16. The method of claim 10, wherein the labeled compound is a small organic molecule, a peptide, a nucleic acid, an oligosaccharide, or a macromolecule.

17. The method of claim 16, wherein the macromolecule is a protein, polysaccharide, DNA, or RNA.

18. The method of claim 16, wherein the compound is labeled with a radioisotope, a fluorescent tag, a bioluminescent tag, or a chemoluminescent tag.

19. The method of claim 18, wherein the radioisotope is ³H, ¹⁴C, ¹²⁵I, or ³²P.

20. The method of claim 16, wherein the labeled compound is a substrate for an enzyme.

21. The method of claim 9, wherein one or more of the assays is an enzyme assay and the substrate has a measurable characteristic.

22. The method of claim 21, wherein the measurable characteristic is UV or visible absorbance or fluorescence.

23. The method of claim 10, wherein the labeled compound interacts with the molecular target, is a substrate for an enzymatic reaction, or alters the function of an ion channel or transporter.

24. The method of claim 9, wherein one or more of the assays is a functional assay.

25. The method of claim 24, wherein binding compounds to the molecular target induces a detectable signal.

26. The method of claim 25, wherein the detectable signal is a chemoluminescent output, a bioluminescent output, a morphological change, or a colorimetric change.

27. The method of claim 24, wherein the assay detects a secondary signal.

28. The method of claim 27, wherein the secondary signal is cAMP, Ca2+ flux, membrane depolarization, IP3 turnover, neurotransmitter release, or ion transport.

29. A method of treating substance abuse, comprising:
(a) determining a set of molecular targets according to the method of claim 2,
(b) providing a database of chemical compounds containing records corresponding to a plurality of chemical compounds, wherein said records include data on the interaction of the chemical compounds with a plurality of molecular targets;
(c) selecting one or more chemical compounds from the database, wherein the selected chemical compound or compounds interact with the molecular targets in a manner substantially the same as the abused or addictive substance; and
(d) administering to a patient in need thereof an effective amount of at least one of the selected chemical compounds.

30. The method according to claim 29, wherein the set of molecular targets is determined according to the method of claim 3.

31. The method of claim 29; wherein one or more assays are used to define the pharmacological activity profile.

32. The method of claim 31, wherein each assay comprises a buffer solution, a cell or tissue preparation containing a molecular target, and a labeled compound that interacts with the molecular target.

33. The method of claim 32, wherein the molecular target is a receptor, transporter, ion channel, or enzyme.

34. The method of claim 32, wherein the wherein the molecular target is from animal tissue, human tissue, or cultured cells.

35. The method of claim 34, wherein the cultured cells natively express the molecular target.

36. The method of claim 34, wherein the cultured cells express a recombinant nucleic acid encoding the molecular target.

37. The method of claim 32, wherein the molecular target is a crude preparation, partially purified, or highly purified.

38. The method of claim 32, wherein the labeled compound is a small organic molecule, a peptide, a nucleic acid, an oligosaccharide, or a macromolecule.

39. The method of claim 36, wherein the macromolecule is a protein, polysaccharide, DNA, or RNA.

40. The method of claim 38, wherein the compound is labeled with a radioisotope, a fluorescent tag, a bioluminescent tag, or a chemoluminescent tag.

41. The method of claim 40, wherein the radioisotope is ³H, ¹⁴C, ¹²⁵I, or ³²P.

42. The method of claim 38, wherein the labeled compound is a substrate for an enzyme.

43. The method of claim 31, wherein one or more of the assays is an enzyme assay and the substrate has a measurable characteristic.

44. The method of claim 43, wherein the measurable characteristic is UV or visible absorbance or fluorescence.

45. The method of claim 32, wherein the labeled compound interacts with the molecular target, is a substrate for an enzymatic reaction, or alters the function of an ion channel or transporter.

46. The method of claim 31, wherein one or more of the assays is a functional assay.

47. The method of claim 46, wherein binding compounds to the molecular target induces a detectable signal.

48. The method of claim 47, wherein the detectable signal is a chemoluminescent output, a bioluminescent output, a morphological change, or a colorimetric change.

49. The method of claim 46, wherein the assay detects a secondary signal.

50. The method of claim 49, wherein the secondary signal is cAMP, Ca2+ flux, membrane depolarization, IP3 turnover, neurotransmitter release, or ion transport.

51. A computer system comprising:
(a) a database containing records corresponding to a plurality of addictive substances, wherein said records include chemoinformatic information, in vivo biochemical information, and information concerning the physiological effects of the addictive substance; and
(b) a user interface allowing a user to view records of the addictive substances.

52. The computer system of claim 51, wherein the information concerning the physiological effects of the addictive substance includes information on the in vivo pharmacology associated with cocaine addiction and dependency.

53. The computer system of claim 51, wherein the chemoinformatic information includes chemical structure, physical chemistry, chemical purity, chemical descriptors or codes, chemical substructure descriptors or codes, solubility, logP, chirality, in vivo biochemical effects, physiological effects, or physiological response information.

54. The computer system of claim 51, further comprising bioinformatic annotations for the molecular targets used for profiling the abused or addictive substance(s).

55. The computer system of claim 54, wherein the bioinformatic annotations include peptide sequence, DNA sequence or links to DNA sequence information for the respective genes, RNA sequence or links to RNA sequence information for the expressed gene transcripts, name, structural class, physiological phenomenon, or pharmacological function information.

56. The computer system of claim 51, wherein relationships between chemicals and biological targets are linked through their in vitro activity and their physiological responses.

57. A method for determining a biological activity profile for cocaine, comprising:
(a) selecting a panel of molecular targets;
(b) defining a pharmacological activity profile for cocaine by;
(i) exposing cocaine to each of the molecular targets in the panel; and
(ii) measuring the ability of cocaine to interact with the molecular targets; and
(c) determining the biological activity profile by identifying in the pharmacological activity profile a subset of the molecular targets whose activity is affected by cocaine.

58. A method for determining a set of one or more molecular targets for developing a treatment for cocaine addiction, comprising:
(a) determining a biological activity profile for cocaine according to the method of claim 57; and
(b) defining the set of molecular targets by identifying those targets in the biological activity profile wherein the interaction between cocaine and the molecular target exceeds a threshold level.

59. The method of claim 58, further comprising utilizing information relating the pathology associated with cocaine addiction to the interaction of cocaine with different molecular targets to define the set of molecular targets.

60. The method of claim 59, wherein the information utilized includes either or both of information concerning at least one positive effect of the abused or addictive substance and information concerning at least one negative effect of the abused or addictive substance.

61. The method of claim 60, wherein the information exists in a relational database.

62. A method for identifying at least one chemical compound to treat cocaine addiction, comprising:
(a) determining a set of molecular targets according to the method of claim 58;
(b) providing a database of chemical compounds containing records corresponding to a plurality of chemical compounds, wherein said records include data on the interaction of the chemical compounds with a plurality of molecular targets; and
(c) selecting one or more chemical compounds from the database, wherein the selected chemical compound or compounds interact with the molecular targets in a manner substantially the same as cocaine.

63. the method according to claim 62, wherein the set of molecular targets is determined according to the method of claim 59.

64. The method of claim 62, wherein one or more assays are used to define the pharmacological activity profile.

65. The method of claim 64, wherein each assay comprises a buffer solution, a cell or tissue preparation containing a molecular target, and a labeled compound that interacts with the molecular target.

66. The method of claim 65, wherein the molecular target is a receptor, transporter, ion channel, or enzyme.

67. The method of claim 65, wherein the wherein the molecular target is from animal tissue, human tissue, or cultured cells.

68. The method of claim 67, wherein the cultured cells natively express the molecular target.

69. The method of claim 67, wherein the cultured cells express a recombinant nucleic acid encoding the molecular target.

70. The method of claim 65, wherein the molecular target is a crude preparation, partially purified, or highly purified.

71. The method of claim 65, wherein the labeled compound is a small organic molecule, a peptide, a nucleic acid, an oligosaccharide, or a macromolecule.

72. The method of claim 71, wherein the macromolecule, is a protein, polysaccharide, DNA, or RNA.

73. The method of claim 71, wherein the compound is labeled with a radioisotope, a fluorescent tag, a bioluminescent tag, or a chemoluminescent tag.

74. The method of claim 73, wherein the radioisotope is ³H, ¹⁴C, ¹²⁵I, or ³²P.

75. The method of claim 71, wherein the labeled compound is a substrate for an enzyme.

76. The method of claim 64, wherein one or more of the assays is an enzyme assay and the substrate has a measurable characteristic.

77. The method of claim 76, wherein the measurable characteristic is UV or visible absorbance or fluorescence.

78. The method of claim 65, wherein the labeled compound interacts with the molecular target, is a substrate for an enzymatic reaction, or alters the function of an ion channel or transporter.

79. The method of claim 64, wherein one or more of the assays is a functional assay.

80. The method of claim 79, wherein binding compounds to the molecular target induces a detectable signal.

81. The method of claim 80, wherein the detectable signal is a chemoluminescent output, a bioluminescent output, a morphological change, or a colorimetric change.

82. The method of claim 79, wherein the assay detects a secondary signal.

83. The method of claim 82, wherein the secondary signal is cAMP, Ca2+ flux, membrane depolarization, IP3 turnover, neurotransmitter release, or ion transport.

84. A method of treating cocaine addiction, comprising:
(a) determining a set of molecular targets according to the method of claim 58;
(b) providing a database of chemical compounds containing records corresponding to a plurality of chemical compounds, wherein said records include data on the interaction of the chemical compounds with a plurality of molecular targets;
(c) selecting one or more chemical compounds from the database, wherein the selected chemical compound or compounds interact with the molecular targets in a manner substantially the same as cocaine; and
(d) administering to a patient in need thereof an effective amount of at least one of the selected chemical compounds.

85. The method according to claim 84, wherein the set of molecular targets is determined according to the method of claim 59.

86. The method of claim 84, wherein one or more assays are used to define the pharmacological activity profile.

87. The method of claim 86, wherein each assay comprises a buffer solution, a cell or tissue preparation containing a molecular target, and a labeled compound that interacts with the molecular target.

88. The method of claim 87, wherein the molecular target is a receptor, transporter, ion channel, or enzyme.

89. The method of claim 87, wherein the wherein the molecular target is from animal tissue, human tissue, or cultured cells.

90. The method of claim 89, wherein the cultured cells natively express the molecular target.

91. The method of claim 89, wherein the cultured cells express a recombinant nucleic acid encoding the molecular target.

92. The method of claim 87, wherein the molecular target is a crude preparation, partially purified, or highly purified.

93. The method of claim 87, wherein the labeled compound is a small organic molecule, a peptide, a nucleic acid, an oligosaccharide, or a macromolecule.

94. The method of claim 93, wherein the macromolecule is a protein, polysaccharide, DNA, or RNA.

95. The method of claim 93, wherein the compound is labeled with a radioisotope, a fluorescent tag, a bioluminescent tag, or a chemoluminescent tag.

96. The method of claim 95, wherein the radioisotope is ³H, ¹⁴C, ¹²⁵I, or ³²P.

97. The method of claim 93, wherein the labeled compound is a substrate for an enzyme.

98. The method of claim 86, wherein one or more of the assays is an enzyme assay and the substrate has a measurable characteristic.

99. The method of claim 98, wherein the measurable characteristic is UV or visible absorbance or fluorescence.

100. The method of claim 81, wherein the labeled compound interacts with the molecular target, is a substrate for an enzymatic reaction, or alters the function of an ion channel or transporter.

101. The method of claim 86, wherein one or more of the assays is a functional assay.

102. The method of claim 101, wherein binding compounds to the molecular target induces a detectable signal.

103. The method of claim 102, wherein the detectable signal is a chemoluminescent output, a bioluminescent output, a morphological change, or a colorimetric change.

104. The method of claim 101, wherein the assay detects a secondary signal.

105. The method of claim 104, wherein the secondary signal is cAMP, Ca2+ flux, membrane depolarization, IP3 turnover, neurotransmitter release, or ion transport.

106. A method of treating cocaine addiction, comprising administering to a patient in need thereof an effective amount of a pharmaceutical composition comprising at least one compound that directly effects the activity of dopamine and serotonin transporters and has substantially no effect on noradrenaline transporters.
